SEQUENCE LISTING (22 sequences)

SEQ ID NO: 1 - DNA, 1669 bp, mammalian
  CDS (43)..(1104)
ttcaaagaggtgacatatttggttctgatcccatctcaagccatgtgtttttgggggatatttttgtgtttgatcttcctggagaaaagttggggacagatacaaatgtcgtgttggcccaagcctttgattccagaacttgagaggcagagatgcaccgttgtaacaccaaaagtcttccgagtcggagaatatgaacaagttacatttgaagcccacggtcacactgacccatttgatgtaaccatctctataaaaagttaccctgataaaaatgctaattactcttcaagctctgtacatttatcaccagaaaataaattcaaaaactctacaatcttaacaattcagcccaaacagttgtctgaagggcaaaactcgtcttcgcatgtgtatttggaagttgtgtccaagcatttttcaacatcaaaaataatgtcaatcgtctatgacaatggcactctcttcattcagactgacaagcctgtgtacactccagagcagcctgtaaaggttgccgtgtattcgctggatgaagccttaaagccagtcaccagagagacagtcttaacgttcatagaccctgaaggatccgaagttggcatagtagaaggaagcaatcatactggaatcacctctttccctgacttcaggattcctactaaccctaagcccggtagatggatgatcaaggctaaatatagagaagatgcttcaacagctggaaccacacactttgaaattaaagagcatgataaagctttcaaaatagccctcgttccaacaagtgatctggaacacccaatggaagaagcacgtggcctgagtctccagccaaaaaagtccctgcaagagatgatacatgagcaagcttcgaaatacaaacatccagtactgaagaaatgttgttatgatggagccagatataaccaccatgaaacctgtgaggaacgagttgcccgtgtgaaaataggcccaaactgtgtcagagccttcagtgaatgctgtgccctggctagcgagaatacctttaagaatatcctcatgtcgcgtcccgatgacagtggatattttactttatctgctaccatactggaaaatgcttaatcttattccctgcaagtatttgaagattacaagtattttctgtgccttcacttttgctggaaactaatgcacaaaatcaaacggagttcatacagcagtgaagcccttccgctgtaactttgccataaatagccttggctgcacggaggtcatttcataaccgtaatttatccactggtctcacaagtgagaccaagctgataaaaacaaattcaccagaagagtttgattgccatgcctagtgaccttgcccatcttcctgtcaggaccctcggtgccctaacatagtagagggtgctcgggggacactcaccgccacaaagaaagctgccatccagccccggagagctgtggagtcaacagcacacaccgtgtgggccaccgtgctgcccaggtgtccataatgctacactaagtcgccacgaataatcagttgtgccagcagagtatgggagccgctaaaggatactatgcttgtaaatgtgtatcacaatcagaatgtttaaatcaataaaatagtattgcccgcgttaaaaaaaaaaaaaaaaaaaaaaaaaaaaaa

SEQ ID NO: 2 - protein, 354 aa, mammalian
MCFWGIFLCLIFLEKSWGQIQMSCWPKPLIPELERQRCTVVTPKVFRVGEYEQVTFEAHGHTDPFDVTISIKSYPDKNANYSSSSVHLSPENKFKNSTILTIQPKQLSEGQNSSSHVYLEVVSKHFSTSKIMSIVYDNGTLFIQTDKPVYTPEQPVKVAVYSLDEALKPVTRETVLTFIDPEGSEVGIVEGSNHTGITSFPDFRIPTNPKPGRWMIKAKYREDASTAGTTHFEIKEHDKAFKIALVPTSDLEHPMEEARGLSLQPKKSLQEMIHEQASKYKHPVLKKCCYDGARYNHHETCEERVARVKIGPNCVRAFSECCALASENTFKNILMSRPDDSGYFTLSATILENA

SEQ ID NO: 3 - DNA, 1170 bp, mammalian
  CDS (21)..(586)
gtcgttggtttcggcggccatggagagcgcagaggagcctctgcccgcgcggccggcgctggagaccgagggcctgaggttcctgcacgtcacagtgggctccctgctggccagctatggctggtacgtcctcttcagctgcatccttctctacattgtcatccagaagctctccgtccgattgagggttttgaggcagaggcagctggaccaggctgacgctgttctggaacctgatgctgttgttaagcgacaagaggctttagccgctgctcgtttgagaatgcaggaagatctaaatgcccaagttgaaaagcataaggaaaaactaagacagcttgaagaagaaaaaaggagacagaagattgaaatgtgggacagcatgcaagaaggcagaagttacagaagaaatccaggaaggcctcaggaagaagatggtcctggaccttctacttcatcatctgtcacccgcaaaggaaaatctgacaaaaagcctttgaggggaaatggttataaccctctgacgggtgaagggggtggaacctgcgcctggagacctggacgcaggggcccatcatctggtggatgaagctaagacccttgttagtgtcgctttgacattagcaaggtgaacccttaaccctcaactcagttgccttacgcacactttcacagtgactagccaaggagaggtggggcttatttccattcgtagctacctgtattctaagggctttggtcagtgtgagctatggacattgtcattaggtcatattctacttagacaacagtcattgatttcatggctacttgctagttgataggttaaaggcctctcgctgtttagcaaacttcataaaggaggcccagtgatgatcctttggggtagaagtccttgctgacaggatggtctctgtgacaggatgcgttcaatgatgtcttccttataaatggtgagcccaccagtgaggattactgatgtgcacagttgatggggtttgcttctgtatatttatttttatgtacagaaatttgcaaaaaaaaataaaaagtaacatttttagcatctttattaaactcaaggaaatttcgttgtgagcttgactttgtctatcagacattaaacagctttttatcattaaaaaaaaaaaaaaaaaaaaaaaaaaaaaa

SEQ ID NO: 4 - protein, 189 aa, mammalian
MESAEEPLPARPALETEGLRFLHVTVGSLLASYGWYVLFSCILLYIVIQKLSVRLRVLRQRQLDQADAVLEPDAVVKRQEALAAARLRMQEDLNAQVEKHKEKLRQLEEEKRRQKIEMWDSMQEGRSYRRNPGRPQEEDGPGPSTSSSVTRKGKSDKKPLRGNGYNPLTGEGGGTCAWRPGRRGPSSGG

SEQ ID NO: 5 - DNA, 1174 bp, mammalian
  CDS (31)..(594)
cagggctgggcggcggcggcggcggcggtcatggaacgccaagaggagtctctgtccgcgcggccggccctggagaccgaggggctgcgcttcctgcacaccacggtgggctccctgctggccacctatggctggtacatcgtcttcagctgcatccttctctacgtggtctttcagaagctttccgcccggctaagagccttgaggcagaggcagctggaccgagctgcggctgctgtggaacctgatgttgttgttaaacgacaagaagctttagcagctgctcgactgaaaatgcaagaagaactaaatgcgcaagttgaaaagcataaggaaaaactgaaacaacttgaagaagaaaaaaggagacagaagattgaaatgtgggacagcatgcaagaaggaaaaagttacaaaggaaatgcaaagaagccccaggaggaagacagtcctgggccttccacttcatctgtcctgaaacggaaatcggacagaaagcctttgcggggaggaggttataacccgttgtctggtgaaggaggcggagcttgctcctggagacctggacgcagaggcccgtcatctggcggatgaggctaagaatcttgttagtgtcacttttgacattagcaagatgaacccttaaccctcgattcaattgccttacgcacgcttttcacagtgactagccaaggggaggtggggttgatttctgttcctaactacacctgcatatgtcagggctccagtcagcaaaaggtatagatgttgcctctaggcatgaggtcattggtcacattctacttggagacagtgattgcattcattgatttcatggttaattgctagttggtaggtaaaggcctctagatgattagcaatcttgataaaagaggcctagtaatgttcttttgaggttagaaatccttgctgctaggacagtctctgtgacaggttgcgttgaatgatgtcttccttatcaatggtgagcccaccagtgaggattactgatgtggacagttgatggggtttgtttctgtatatttatttttatgtacagaactttgtaaaaacgaaactatttaaaaaacaagaataacatttttagcatctttattcaaggagatttatggacttcaatttgtctatcaaacattaaatagctttttattac

SEQ ID NO: 6 - protein, 187 aa, mammalian
MERQEESLSARPALETEGLRFLHTTVGSLLATYGWYIVFSCILLYVVFQKLSARLRALRQRQLDRAAAAVEPDVVVKRQEALAAARLKMQEELNAQVEKHKEKLKQLEEEKRRQKIEMWDSMQEGKSYKGNAKKPQEEDSPGPSTSSVLKRKSDRKPLRGGGYNPLSGEGGGACSWRPGRRGPSSGG

SEQ ID NO: 7 - DNA, 279 bp, mammalian
  CDS (54)..(140)
ctgaaaaggctgttgtcaagatggagtgtctaacccagtaatccaaggaccaaatgctgagtccacacagtgtggccagcatgctgtctgcagttgaagcagggacagtttttcttctagtgactagcttaccacattgaggcaaactccatgtggaggttcttcgatgctcatcatcttctttgaagtggagtggagacgctgccagaagcagacgtgtttcactggtcaagaaagccttttattaataaaacatctcaaatgccataaaaaaaaaaa

SEQ ID NO: 8 - protein, 28 aa, mammalian
MLSPHSVASMLSAVEAGTVFLLVTSLPH

SEQ ID NO: 9 - DNA, 5251 bp, mammalian
cagggctgggcggcggcggcggcggcggtcatggaacgccaagaggagtctctgtccgcgcggccggccctggagaccgaggggctgcgcttcctgcacaccacgggtgagtcgttgcggggcagccgggcgcgcgccgccacttttgcgacgcgcagccatgatgggtgggtcgtccgccgctgcaccgggcgccggagcctgggaggcctgggaacggtcgggcgttggcgcttacgcggaccttgggcagcaggcccggaccttgcgcggaggcttctcgggagccgcacttccctgggcggctcggctgtcccttgtttgcgcaagtcttttttgcgaaccaagcccttcctgtggtagttactggggtcactcggccgttggcgtttgcctctgggacccgtcccacacagccccatacacactcctgactccccgcgctgtcacccctttctatgtggctctgaaaggcctttgccttcctgattcagattagttgctcttcattcttcaaaacccagttgctgtgccctccacactctaactgcccccgactccccagatggttgggaagtctcacttctcagtgatccctgaattgtcgcacttcttgagttcgtgttttaacgatctacttaggaggctttttcctcagcctagaccatgaaggctttgagggcaggagttacactttgtgtttgttgagtcttatggaaaggtcaactagtagtgtcatttttagttttttgaaaactgtttttcttttcagtgggctccctgctggccacctatggctggtacatcgtcttcagctgcatccttctctacgtggtctttcagaagctttccgcccggctaagagccttgaggcagaggcagctggaccgagctgcggctgctgtgggttagtgcctgataaccgaaatgaaagcggtggttttgcacctcctttatattaagagttagtctcttagtaaaagtaagaggggccacacaggaagaccctgtctctatttaaaaaaaaaaaaaatagccgggagtggcggcacgcacctgtagtcccagctgctcaggaggctgaggcgggataatcacttgagtccagggagtcaaagctgcagtgggctatgctcgggccacactacactccagcctgggcaattgattgagaccttgtctttaaaaaaaaaaaaaaaaaaaaagtaggaagtatatggttctcggtggggcgcggtggctcacacctgtaatcccagcactttgggaagccgaggcaggaggatgacttgaggtcaggggttcgagaacagcctggccaacatggtgaaaccctgtctctactaaaaatacaaatattagtggggcgtggtgacgggcacctgtaatcccagctattagggtggctgaggcaggagaaatcgcttgaacctgggagctggagattgcagtgagctgagattgtgccactgcactccagcctgggcaacagagtgagactgtcttttctttcttttttttttttttttctatgagatggagtctagccttgttgcaaagagcgagactctatgagtagacgttatgaatagaaatgagttcatttctattcataatgctatttggaaggatttttcttttctgtagaaacaaatacttaagaatcttctgcgctaattaagggatggataatgatttagaaaactttatatttccttggtagtcttccaggattctagtcagcctagagactgtgggtgtcactgaggtatccaagatgtgctctgtgtggccactatcccaggctttatgaatcggaattgctcaggggaactcagaaattggcatttctaacagatttctggtgatgtagatatttcgggctaaaatccgtggctcagcaacagacccctgccccctgaagcagtaaaatgtatgcagaggggttaggagtacttatgtaaaaatatgttgtttcattgtctgatatccatacctctttatacttttaataatatggacactcaaaagtttctattttatattgtacacagtgctttatctccatttttttctgacattttagaacctgatgttgttgttaaacgacaagaagctttagcagctgctcgactgaaaatgcaagaagaactaaatgcgcaagttgaaaagcataaggaaaaactgaaacaagtatgaactggtttcagtttgaatgtgtgcatagaaattgtctgaggtttagtggctaacgatgcctgtgtctgtgttgtctataagcttctaggaccaggtcctatcccattagattcaataagcatttcagttcctaccatgtaagtattggtgatatcaagaagaatacacgattgttagggaacactagatgtgtgaatatattaccatgaaaggtccagagcacaaaaggagggacaggctggagcagggagcatgtgagtgtgtgtgtgcatgtgcctgtgtcttccccattaccaaaaatgtcctgacaggagtgagtttcagaagaatggagtcagtaatctttttcatgaaacattttgctttctttaatagtgtacaaaaaccaaagctgctctatgtgagttaaactcacactaccagatcacaacagttttattaactaaagaaaacgagggtgaagtttgttctgaaagacatttaaattaagaattatcagagttagctttgtctttgagagaaatggcagcttctgaattctttctgtaaaatgtgattgtttctcagcttgaagaagaaaaaaggagacagaagattgaaatgtgggacagcatgcaagaaggaaaaagttacaaaggaaatgcaaagaagccccaggtgactggagacctcggccggctggcatgcggtagatgaagattgccaagtagaatgttttaattgcttcttacactactgtgtgtgttcaaacaggaggaagacagtcctgggccttccacttcatctgtcctgaaacggaaatcggacagaaagcctttgcggggaggaggtaagcaccactgatgtcaaatgttaacagattttcaacacttacaggatatagttaccttttaagaacaagattgtttgtttctttgtccataaattaagactaattccttaggattgtgaagattcaataaaggaaacagatgcaaatcacctcctaggtcctcactaagtacttagaaggattgtacttatagtattctaacttgatccttctgcagccccgtagagggagagctaagtagggtgaggaattgtctgccaatcttcagatgagtgtcaaggagctggaacacagtggttttggtctttctggctgggaccaccttgtttcttgcaaataacaaggagtagcagacagatgctcatccaaagctgcttcctgtgtgcagcactgccccggggactctggatgatgccacagcagtctgtcttcatcccatccctgagaatttcaaatctgggaagatgggactcacaaacgaaaataagcaatccttggtgattctggctaagagttgcaagttactgctgaggaaggaaagaacaaacacactagaacactgtaggaaccaaggcggaagattttgtatcctccataggaggagaggggcaccgcagaggccctgatggtgtctttgaggactgaggaaagactggggcatgggctccaaggcagcagggccacagacttggctgaccttaaacgctgagctgtaatcccctttgtgtcagaagactaaacctggcttgctgtagagaaggtgatgcatctggaaagaaaatgctatttttaaatggtcctgccggaagcttatttttagacacatagaggtgatatttaggagaggaatggaaatcgtagaagatggaatgcagggtgtgcttgcctgcacggcctctttcagcatccccagcatttctgagctgggacttttgactagcctggctttacaaataaggaaactgaggcacagtgtttaattgcccaaagattccactataagtaaggagtaaaagtaacatttaagttctgggtggccctagaaccttagcactcaaccaggttaccagttgtgcactgactttgggaagctcatgagggagtggggtggttgggggtagggaaggatacagaagaccccgttctgactggtagaagtgacaagtttgactcttgattttttttaatctgttttctgtagcgtgaacagcccttatttgaatgtatgagttttagtaagcactgtgataggaggattcatatacttaaatcaggccctcttgagagagttttttggtgacccttttgcatgtgtttcggaggttgggacaaagaagctgaatgacttttttccccaccagacaatcagttcaaatggcaatcacaatataaaggtttttttttttttcacatagctaaaaggtttttttaaatgtcccttaggatctgtatctttgcagtgctttgcgtgtcactctcataattttattgtggatatacaatgttcccagattttcagatttttatcaatactgttgtgctgcttttctgtcctcccaggttataacccgttgtctggtgaaggaggcggagcttgctcctggagacctggacgcagaggcccgtcatctggcggatgaggctaagaatcttgttagtgtcacttttgacattagcaagatgaacccttaaccctcgattcaattgccttacgcacgcttttcacagtgactagccaaggggaggtggggttgatttctgttcctaactacacctgcatatgtcagggctccagtcagcaaaaggtatagatgttgcctctaggcatgaggtcattggtcacattctacttggagacagtgattgcattcattgatttcatggttaattgctagttggtaggtaaaggcctctagatgattagcaatcttgataaaagaggcctagtaatgttcttttgaggttagaaatccttgctgctaggacagtctctgtgacaggttgcgttgaatgatgtcttccttatcaatggtgagcccaccagtgaggattactgatgtggacagttgatggggtttgtttctgtatatttatttttatgtacagaactttgtaaaaacgaaactatttaaaaaacaagaataacatttttagcatctttattcaaggagatttatggacttcaatttgtctatcaaacattaaatagctttttattac

SEQ ID NO: 10 - DNA, 21 bp, mammalian
gggagagctgtggagtcaaca

SEQ ID NO: 11 - DNA, 22 bp, mammalian
cgtggcgacttagtgtagcatt

SEQ ID NO: 12 - DNA, 23 bp, mammalian
gatgcgttcaatgatgtcttcct

SEQ ID NO: 13 - DNA, 22 bp, mammalian
agaagcaaaccccatcaactgt

SEQ ID NO: 14 - DNA, 21 bp, mammalian
tggaggttcttcgatgctcat

SEQ ID NO: 15 - DNA, 22 bp, mammalian
cagtgaaacacgtctgcttctg

SEQ ID NO: 16 - DNA, 22 bp, mammalian
gcaaagacctgtatgccaacac

SEQ ID NO: 17 - DNA, 23 bp, mammalian
gccagagcagtgatctctttctg

SEQ ID NO: 18 - DNA, 21 bp, mammalian
accgtgctgcccaggtgtcca

SEQ ID NO: 19 - DNA, 29 bp, mammalian
tgagcccaccagtgaggattactgatgtg

SEQ ID NO: 20 - DNA, 29 bp, mammalian
atcttctttgaagtggagtggagacgctg

SEQ ID NO: 21 - DNA, 25 bp, mammalian
tccggtccacaatgcctgggtacat

SEQ ID NO: 22 - protein, 18 aa, mammalian
RPQEEDGPGPSTSSSVTR

(19) United States
(12) Patent Application Publication
(10) Pub. No.: US 2002/0169287 A1
(43) Pub. Date: Nov. 14, 2002

(54) Novel genes and their use in the modulation of obesity, diabetes and energy imbalance

(75) Inventors: Gregory Collier, Ocean Grove (AU); Paul Zev Zimmet, Toorak (AU); Kenneth Russell Walder, Ocean Grove (AU); Kelly Fiona Windmill, Newtown (AU); Janine Susan McMillan, Torquay (AU)

Correspondence Address: Leopold Presser, Scully, Scott, Murphy & Presser, 400 Garden City Plaza, Garden City, NY, 11530, US

(73) Assignee: AUTOGEN RESEARCH PTY LTD., MELBOURNE (AU)

(21) Appl. No.: 10/039,050

(22) Filed: Dec. 31, 2001

Related U.S. Application Data
(63) Continuation of application No. PCT/AU00/00786, filed on Jun. 29, 2000.

(51) Int. Cl. C07K 14/435; C07H 21/04; C12P 21/02; C12N 5/06

(52) U.S. Cl. 530/350; 536/23.5; 435/69.1; 435/325; 435/320.1

(57) ABSTRACT

The present invention relates generally to nucleic acid molecules encoding proteins associated with the modulation of obesity, diabetes and/or metabolic energy levels. More particularly, the present invention is directed to nucleic acid molecules and the recombinant and purified proteins encoded thereby and their use in therapeutic and diagnostic protocols for conditions such as obesity, diabetes and energy imbalance. The subject nucleic acid molecules and proteins and their derivatives, homologs, analogs, chemical equivalents and mimetics are proposed as therapeutic and diagnostic agents for obesity, diabetes and energy imbalance.

### CROSS REFERENCE TO RELATED APPLICATION

This application a continuation of International Application No. PCT/AU00/00786, filed on Jun. 29, 2000.

### FIELD OF THE INVENTION

The present invention relates generally to nucleic acid molecules encoding proteins associated with the modulation of obesity, diabetes and/or metabolic energy levels. More particularly, the present invention is directed to nucleic acid molecules and the recombinant and purified proteins encoded thereby and their use in therapeutic and diagnostic protocols for conditions such as obesity, diabetes and energy imbalance. The subject nucleic acid molecules and proteins and their derivatives, homologs, analogs, chemical equivalents and mimetics are proposed as therapeutic and diagnostic agents for obesity, diabetes and energy imbalance.

### BACKGROUND OF THE INVENTION

Bibliographic details of the publications referred to by author in this specification are collected at the end of the description.

The reference to any prior art in this specification is not, and should not be taken as, an acknowledgement or any form of suggestion that prior art forms part of the common general knowledge in Australia.

Obesity is defined as the pathological condition of increased body fat content and is thought to result from the sustained imbalance between energy intake and energy expenditure. The incidence of this metabolic disorder is high, affecting approximately 23% of adult Americans (Flegal et al. 1998).

The high incidence of obesity amounts to a serious public health problem due to the increased risk of complications such as cardiovascular disease, type 2 diabetes and certain types of cancer (Bouchard 1994). Type 2 diabetes may be defined as a pathological increase in blood glucose concentration. It characteristically develops in obese, middle-aged individuals and, if not adequately controlled, leads to the onset of complications such as blindness, renal failure and peripheral vascular insufficiency. As with obesity, type 2 diabetes is highly prevalent in both affluent and developing socieites, with an estimated prevalence rate of 5-10% in adult Americans (Harris et al. 1998).

The prevalence rates of both obesity and type 2 diabetes continue to increase worldwide (Bennet and Magnus 1994; Bouchard 1994; Flegal et al. 1998; Harris et al. 1998). In addition, certain ethnic (e.g. Native American, Australian Aborigines, Pacific Islanders) and socioeconomic groups (low income) appear to be particularly susceptible to the onset of obesity and diabetes (Zimmet et al. 1995; Harris et al. 1998; Martikainen and Marmot 1999; Story et al. 1999). The public health impacts of obesity and type 2 diabetes onset are reflected by the high cost burden imposed by these diseases. It has been estimated that type 2 diabetes alone accounts for 2-3% of the total health care budget in every country worldwide (Jonsson 1998), costing about US$40 billion annually in the USA alone (Bouchard 1994). In addition, the indirect costs of type 2 diabetes have been estimated using “disability-adjusted life-years” (DALYs). In 1990, 7.97 million DALYs were lost due to type 2 diabetes onset. Similarly, obesity imposes a substantial economic burden on society both directly and indirectly through the close relationship between obesity and its complications such as cardiovascular disease and type 2 diabetes.

Obesity and type 2 diabetes are both systemic diseases with ill-defined etiology and pathophysiology. However, several tissues have been implicated in the disease processes including the hypothalamus, liver and adipose tissue. The hypothalamus plays a central role in energy balance and factors produced by and/or acting on the hypothalamus have been extensively investigated. These factors include neuropeptide Y, corticotropin-releasing factor, melanin-concentrating hormone, leptin and many other proteins which affect food intake in experimental animal models. It has been proposed that genetic alterations perturbing the metabolic pathways that regulate energy balance in the hypothalamus could contribute to the development of obesity, and subsequently diabetes.

The liver is thought to play a significant role in carbohydrate metabolism, as it is the only organ in which glucose is produced. It is also a major site of glucose storage in the form of glycogen. Alteration in the output of glucose from the liver (“elevated hepatic glucose output”) is an early pathological event in the development of type-2 diabetes, and together with reduced clearance of glucose from the blood, is a significant contributor to the rise in blood glucose concentration which is characteristic of type 2 diabetes. In addition, the liver is a large organ and alterations in the metabolic activity of the liver may contribute to overall variations in whole body energy expenditure.

Adipose tissue is the site of fat storage for the body, and is the principal organ involved in the development of obesity as it is the site of excess fat storage. Previously thought to be rather metabolically inert, recent studies have shown that a number of factors are secreted from adipose tissue, which factors may act to regulate energy balance and other metabolic processes. For example, leptin is secreted by adipose tissue and is thought to act on the hypothalamus to reduce food intake and increase energy expenditure (Zhang et al. 1994). It is considered likely that other factors produced by adipocytes may act either locally or systemically to regulate energy balance.

In work leading up to the present invention the inventors have identified novel genes which are differentially expressed in association with obesity, diabetes and energy metabolism. The identification of these genes permits the rational design of drugs for the modulation of the functional activity of these genes and the further identification of a range of molecules for use in therapy, diagnosis, antibody generation and modulation of obesity, diabetes or energy metabolism.

### SUMMARY OF THE INVENTION

The subject specification contains nucleotide and amino acid sequence information prepared using the programme PatentIn Version 2.0, presented herein after the bibliography. Each nucleotide or amino acid sequence is identified in the sequence listing by the numeric indicator <210>followed by the sequence identifier (e.g. <210>1, <210>2, etc). The length, type of sequence (DNA, protein (PRT), etc) and source organism for each nucleotide or amino acid sequence are indicated by information provided in the numeric indicator fields <211>, <212>and <213>, respectively. Nucleotide and amino acid sequences referred to in the specification are defined by the information provided in numeric indicator field <400>followed by the sequence identifier (eg. <400>1, >400>2, etc). A summary of the sequences with given SEQ ID NOS is provided before the Examples.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

One aspect of the present invention provides an isolated nucleic acid molecule or derivative, homologue or analogue thereof comprising a nucleotide sequence encoding or complementary to a sequence encoding a protein or a derivative, homologue or mimetic of said protein wherein said nucleic acid molecule is differentially expressed in liver tissue of obese animals compared to lean animals.

Another aspect of the present invention provides an isolated nucleic acid molecule or derivative, homologue or analogue thereof comprising a nucleotide sequence encoding or complementary to a sequence encoding a protein or a derivative, homologue or mimetic of said protein wherein said nucleic acid molecule is differentially expressed in liver tissue of fed animals compared to fasted animals.

Yet another aspect of the present invention provides an isolated nucleic acid molecule or derivative, homologue or analogue thereof comprising a nucleotide sequence encoding, or a nucleotide sequence complementary to a nucleotide sequence encoding, an amino acid sequence substantially as set forth in <400>2 or a derivative, homologue or mimetic thereof or having at least about 45% similarity to at least 10 contiguous amino acids in <400>2.

Still another aspect of the present invention contemplates an isolated nucleic acid molecule or derivative, homologue or analogue thereof comprising a nucleotide sequence substantially as set forth in <400>1 or a derivative or homologue thereof, or capable of hybridising to <400>1 under low stringency conditions.

Still yet another aspect of the present invention contemplates a nucleic acid molecule or a derivative, homologue or analogue thereof comprising a nucleotide sequence substantially as set forth in <400>1 or a derivative or homologue thereof or capable of hybridising to <400>1 under low stringency conditions and which encodes an amino acid sequence corresponding to an amino acid sequence set forth in <400>2 or a sequence having at least about 45% similarity to at least 10 contiguous amino acids in <400>2.

Yet still another aspect of the present invention contemplates a nucleic acid molecule comprising a sequence of nucleotides substantially as set forth in <400>1.

A further aspect of the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>4 or a derivative, homologue or mimetic thereof or having at least about 45% similarity to at least 10 contiguous amino acids in <400>4.

Another further aspect of the present invention contemplates a nucleic acid molecule or derivative, homologue or analogue thereof comprising a nucleotide sequence substantially as set forth in <400>3 or a derivative or homologue thereof, or capable of hybridising to <400>3 under low stringency conditions.

Still another further aspect of the present invention contemplates a nucleic acid molecule or derivative, homologue or analogue thereof comprising a nucleotide sequence substantially as set forth in <400>3 or a derivative or homologue thereof or capable of hybridising to <400>3 under low stringency conditions and which encodes an amino acid sequence corresponding to an amino acid sequence set forth in <400>4 or a sequence having at least about 45% similarity to at least 10 contiguous amino acids in <400>4.

Yet another further aspect of the present invention contemplates a nucleic acid molecule comprising a sequence of nucleotides substantially as set forth in <400>3.

Still yet another further aspect of the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>6 or a derivative, homologue or mimetic thereof or having at least about 45% similarity to at least 10 contiguous amino acids in <400>6.

Yet still another further aspect of the present invention contemplates a nucleic acid molecule or derivative, homologue or analogue thereof comprising a nucleotide sequence substantially as set forth in <400>5 or a derivative or homologue thereof, or capable of hybridising to <400>5 under low stringency conditions.

Another aspect of the present invention contemplates a nucleic acid molecule or derivative, homologue or analogue thereof comprising a nucleotide sequence substantially as set forth in <400>5 or a derivative, homologue or mimetic thereof or capable of hybridising to <400>5 under low stringency conditions and which encodes an amino acid sequence corresponding to an amino acid sequence set forth in <400>6 or a sequence having at least about 45% similarity to at least 10 contiguous amino acids in <400>6.

Yet another aspect of the present invention contemplates a nucleic acid molecule comprising a sequence of nucleotides substantially as set forth in <400>5.

Still yet another aspect of the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>8 or a derivative, homologue or mimetic thereof or having at least about 45% similarity to at least 10 contiguous amino acids in <400>8.

Still another aspect of the present invention contemplates a nucleic acid molecule or derivative, homologue or analogue thereof comprising a nucleotide sequence substantially as set forth in <400>7 or a derivative or homologue thereof, or capable of hybridising to <400>7 under low stringency conditions.

A further aspect of the present invention contemplates a nucleic acid molecule or derivative, homologue or analogue thereof comprising a nucleotide sequence substantially as set forth in <400>7 or a derivative, homologue or mimetic thereof or capable of hybridising to <400>7 under low stringency conditions and which encodes an amino acid sequence corresponding to an amino acid sequence set forth in <400>8 or a sequence having at least about 45% similarity to at least 10 contiguous amino acids in <400>8.

Another further aspect of the present invention contemplates a nucleic acid molecule comprising a sequence of nucleotides substantially as set forth in <400>7.

In yet another further aspect, the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>6 or a derivative, homologue or mimetic thereof or having at least about 45% similarity to at least 10 contiguous amino acids in <400>6.

Still another further aspect of the present invention contemplates a nucleic acid molecule or derivative, homologue or analogue thereof comprising a nucleotide sequence substantially as set forth in <400>9 or a derivative or homologue thereof, or capable of hybridising to <400>9 under low stringency conditions.

Yet another further aspect of the present invention contemplates a nucleic acid molecule or derivative, homologue or analogue thereof comprising a nucleotide sequence substantially as set forth in <400>9 or a derivative, homologue or mimetic thereof or capable of hybridising to <400>9 under low stringency conditions and which encodes an amino acid sequence corresponding to an amino acid sequence set forth in <400>6 or a sequence having at least about 45% similarity to at least 10 contiguous amino acids in <400>6.

Still yet another further aspect of the present invention contemplates a nucleic acid molecule comprising a sequence of nucleotides substantially as set forth in <400>9.

Another aspect of the present invention contemplates a genomic nucleic acid molecule or derivative homologue or analogue thereof capable of hybridising to <400>1 or a derivative or homologue thereof under low stringency conditions at 42° C.

Yet another aspect of the present invention contemplates a genomic nucleic acid molecule or derivative homologue or analogue thereof capable of hybridising to <400>3 or a derivative or homologue thereof under low stringency conditions at 42° C.

Still another aspect of the present invention contemplates a genomic nucleic acid molecule or derivative homologue or analogue thereof capable of hybridising to <400>7 or a derivative or homologue thereof under low stringency conditions at 42° C.

Still yet another aspect of the present invention contemplates a genomic nucleic acid molecule or derivative homologue or analogue thereof capable of hybridising to <400>5 or a derivative or homologue thereof under low stringency conditions.

Yet another aspect of the present invention contemplates a cDNA nucleic acid molecule or derivative, homologue or analogue thereof capable of hybridising to <400>9 or a derivative or homologue thereof under low stringency conditions.

In another aspect the nucleotide sequence corresponding to B38 is a cDNA sequence comprising a sequence of nucleotides as set forth in <400>1 or a derivative, homologue or analogue thereof including a nucleotide sequence having similarity to <400>1.

In still another aspect the nucleotide sequence corresponding to B55 is a cDNA sequence comprising a sequence of nucleotides as set forth in <400>3 or a derivative, homologue or analogue thereof including a nucleotide sequence having similarity to <400>3.

In yet another aspect the nucleotide sequence corresponding to B55 is a cDNA sequence comprising a sequence of nucleotides as set forth in <400>5 or a derivative homologue or analogue thereof including a nucleotide sequence having similarity to <400>5.

In still yet another aspect the nucleotide sequence corresponding to B55 is a genomic sequence comprising a sequence of nucleotides as set forth in <400>9 or a derivative homologue or analogue thereof including a nucleotide sequence having similarity to <400>9.

In yet a further aspect of the nucleotide sequence corresponding to B60 is a cDNA sequence comprising a sequence of nucleotides as set forth in <400>7 or a derivative, homologue or analogue thereof including a nucleotide sequence having similarity to <400>7.

A derivative of the nucleic acid molecule of the present invention also includes a nucleic acid molecule capable of hybridising to a nucleotide sequence as set forth in any one or more of <400>1, >400>3, >400>5, >400>7 or <400>9 under low stringency conditions. Preferably, low stringency is at 42° C.

Another aspect of the present invention is directed to an isolated protein selected from the list consisting of:

(i) a protein encoded by a novel nucleic acid molecule which molecule is differentially expressed in liver tissue of obese animals compared to lean animals or a derivative, homologue, analogue, chemical equivalent or mimetic thereof.

(ii) a protein encoded by a novel nucleic acid molecule which molecule is differentially expressed in liver tissue of fed animals compared to fasted animals or a derivative, homologue, analogue, chemical equivalent or mimetic thereof.

(iii) B38, B55 or B60 or a derivative, homologue, analogue, chemical equivalent or mimetic thereof.

(iv) a protein having an amino acid sequence substantially as set forth in <400>2 or a derivative, homologue or mimetic thereof or a sequence having at least about 45% similarity to at least 10 contiguous amino acids in <400>2 or a derivative, homologue, analogue, chemical equivalent or mimetic of said protein.

(v) a protein having an amino acid sequence substantially as set forth in <400>4 or a derivative, homologue or mimetic thereof or a sequence having at least about 45% similarity to at least 10 contiguous amino acids in <400>4 or a derivative, homologue, analogue, chemical equivalent or mimetic of said protein.

(vi) a protein having an amino acid sequence substantially as set forth in <400>6 or a derivative, homologue or mimetic thereof or a sequence having at least about 45% similarity to at least 10 contiguous amino acids in <400>6 or a derivative, homologue, analogue, chemical equivalent or mimetic of said protein.

(vii) a protein having an amino acid sequence substantially as set forth in <400>8 or a derivative, homologue or mimetic thereof or a sequence having at least about 45% similarity to at least 10 contiguous amino acids in <400>8 or a derivative, homologue, analogue, chemical equivalent or mimetic of said protein.

(viii) a protein encoded by a nucleotide sequence substantially as set forth in <400>1 or a derivative, homologue or analogue thereof or a sequence encoding an amino acid sequence having at least about 45% similarity to at least 10 contiguous amino acids in <400>2 or a derivative, homologue, analogue, chemical equivalent or mimetic of said protein.

(ix) a protein encoded by a nucleotide sequence substantially as set forth in <400>3 or a derivative, homologue or analogue thereof or a sequence encoding an amino acid sequence having at least about 45% similarity to at least 10 contiguous amino acids in <400>4 or a derivative, homologue, analogue, chemical equivalent or mimetic of said protein.

(x) a protein encoded by a nucleotide sequence substantially as set forth in <400>5 or a derivative, homologue or analogue thereof or a sequence encoding an amino acid sequence having at least about 45% similarity to at least 10 contiguous amino acids in <400>6 or a derivative, homologue, analogue, chemical equivalent or mimetic of said protein.

(xi) a protein encoded by a nucleotide sequence substantially as set forth in <400>7 or a derivative, homologue or analogue thereof or a sequence encoding an amino acid sequence having at least about 45% similarity to at least 10 contiguous amino acids in <400>8 or a derivative, homologue, analogue, chemical equivalent or mimetic of said protein.

(xii) a protein encoded by a nucleotide sequence substantially as set forth in <400>9 or a derivative, homologue or analogue thereof or a sequence encoding an amino acid sequence having at least about 45% similarity to at least 10 contiguous amino acids in <400>6 or a derivative, homologue, analogue, chemical equivalent or mimetic of said protein.

(xiii) a protein encoded by a nucleic acid molecule capable of hybridising to the nucleotide sequence as set forth in <400>1 or a derivative, homologue or analogue thereof under low stringency conditions and which encodes an amino acid sequence substantially as set forth in <400>2 or a derivative, homologue or mimetic thereof or an amino acid sequence having at least about 45% similarity to at least 10 contiguous amino acids in <400>2.

(xiv) a protein encoded by a nucleic acid molecule capable of hybridising to the nucleotide sequence as set forth in <400>3 or a derivative, homologue or analogue thereof under low stringency conditions and which encodes an amino acid sequence substantially as set forth in <400>4 or a derivative, homologue or mimetic thereof or an amino acid sequence having at least about 45% similarity to at least 10 contiguous amino acids in <400>4.

(xv) a protein encoded by a nucleic acid molecule capable of hybridising to the nucleotide sequence as set forth in <400>5 or a derivative, homologue or analogue thereof under low stringency conditions and which encodes an amino acid sequence substantially as set forth in <400>6 or a derivative, homologue or mimetic thereof or an amino acid sequence having at least about 45% similarity to at least 10 contiguous amino acids in <400>6.

(xvi) a protein encoded by a nucleic acid molecule capable of hybridising to the nucleotide sequence as set forth in <400>7 or a derivative, homologue or analogue thereof under low stringency conditions and which encodes an amino acid sequence substantially as set forth in <400>8 or a derivative, homologue or mimetic thereof or an amino acid sequence having at least about 45% similarity to at least 10 contiguous amino acids in <400>8.

(xvii) a protein encoded by a nucleic acid molecule capable of hybridising to the nucleotide sequence as set forth in <400>9 or a derivative, homologue or analogue thereof under low stringency conditions and which encodes an amino acid sequence substantially as set forth in <400>6 or a derivative, homologue or mimetic thereof or an amino acid sequence having at least about 45% similarity to at least 10 contiguous amino acids in <400>6.

(xviii) a protein as defined in any one of paragraphs (i) to (xvii) in a homodimeric form. (xix) a protein as defined in any one of paragraphs (i) to (xvii) in a heterodimeric form.

The present invention contemplates therapeutic and prophylactic uses of B38, B55 and B60 amino acid and nucleic acid molecules, in addition to B38, B55 and B60 agonistic and antagonistic agents.

The present invention contemplates a method of modulating expression of B38, B55 and/or B60 in a mammal, said method comprising contacting the B38, B55 and/or B60 gene with an effective amount of an agent for a time and under conditions sufficient to upregulate, downregulate or otherwise modulate expression of B38, B55 and/or B60.

Another aspect of the present invention contemplates a method of modulating activity of B38, B55 and/or B60 in a subject, said method comprising administering to said subject a modulating effective amount of an agent for a time and under conditions sufficient to increase or decrease B38, B55 and/or B60 activity.

Still another aspect of the present invention relates to a method of treating a mammal suffering from a condition characterised by one or more symptoms of obesity, anorexia, diabetes and/or energy imbalance said method comprising administering to said mammal an effective amount of an agent for a time and under conditions sufficient to modulate the expression of B38, BSS and/or B60 or sufficient to modulate the activity of B38, B55 and/or B60.

In another aspect the present invention relates to a method of treating a mammal suffering from a disease condition characterised by one or more symptoms of obesity, anorexia, diabetes or energy imbalance said method comprising administering to said mammal an effective amount of B38, B55 and/or B60 or B38, B55 and/or B60.

In another aspect, the present invention contemplates a pharmaceutical composition comprising a modulator of B38, B55 and/or B60 expression or B38, B55 and/or B60 activity and one or more pharmaceutically acceptable carriers and/or diluents.

In yet another aspect the pharmaceutical composition comprises B38, B55 and/or B60 or B38, BS5 and/or B60 or a derivative, homologue, analogue, chemical equivalent or mimetic thereof and one or more pharmaceutically acceptable carriers and/or diluents.

Still another aspect of the present invention is directed to antibodies to B38, B55 and/or B60 or B38, B55 and/or B60 including catalytic antibodies.

Yet another aspect of the present invention contemplates a method for detecting B38, B55 and/or B60 or B38, B55 and/or B60 mRNA in a biological sample from a subject said method comprising contacting said biological sample with an antibody specific for B38, B55 and/or B60 or B38, B55 and/or B60 mRNA or its derivatives or homologs for a time and under conditions sufficient for a complex to form, and then detecting said complex. Such methods may be particularly useful for the diagnosis of the development of or predisposition to obesity, anorexia, diabetes or energy imbalance.

### BRIEF DESCRIPTION OF THE DRAWINGS

FIG. 1 is a schematic representation of the amino acid sequence of B55 in the Israeli sand rat (ISR), mouse, rat and human. Mouse, rat and human sequences were deduced from 3, 5 and 8 expressed sequence tags (ESTs), respectively. No rat EST was found which covered the 5′ and 3′ region of the protein. Dashes indicate homology to the ISR sequence, and forward slashes indicate a deletion.

FIG. 2 is a graphical representation of the levels of B55 gene expression in the liver and adipose tissue of fed and fasted animals of groups A, B and C. Gene expression levels were determined by Real Time PCR of cDNA, relative to the house-keeping gene β-actin.

FIG. 3 is a graphical representation of B60 gene expression in the liver versus body weight with all animals together and in individual groups (top) and B60 gene expression in the muscle of fasted animals versus body weight and insulin (bottom). Gene expression levels were determined by Real Time PCR of cDNA, relative to the house-keeping gene β-actin.

FIG. 4 is a graphical representation of B38 gene expression in the liver versus body weight with all animals together and in individual groups (top) and B38 gene expression in the liver and adipose tissue versus blood triglyceride levels. Gene expression levels were determined by Real Time PCR of cDNA, relative to the house-keeping gene β-actin.

FIG. 5 is a schematic representation of the genomic structure of the human B55 gene.

FIG. 6 is a schematic representation of the human B55 gene <400>9 showing the transcription initiation and termination sites and the intron/exon boundaries.

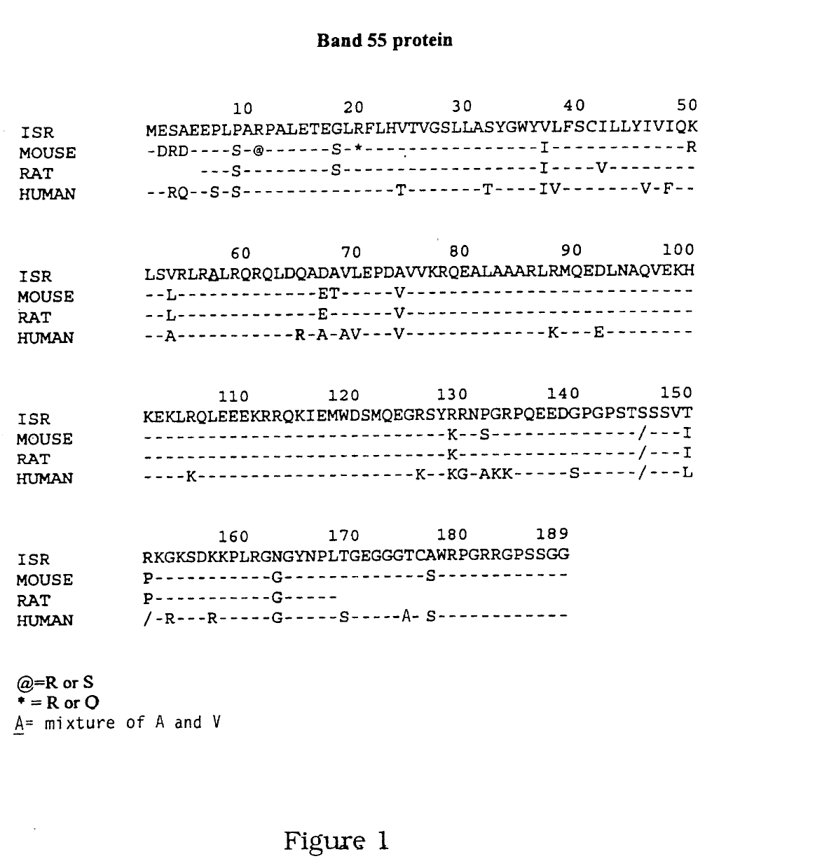

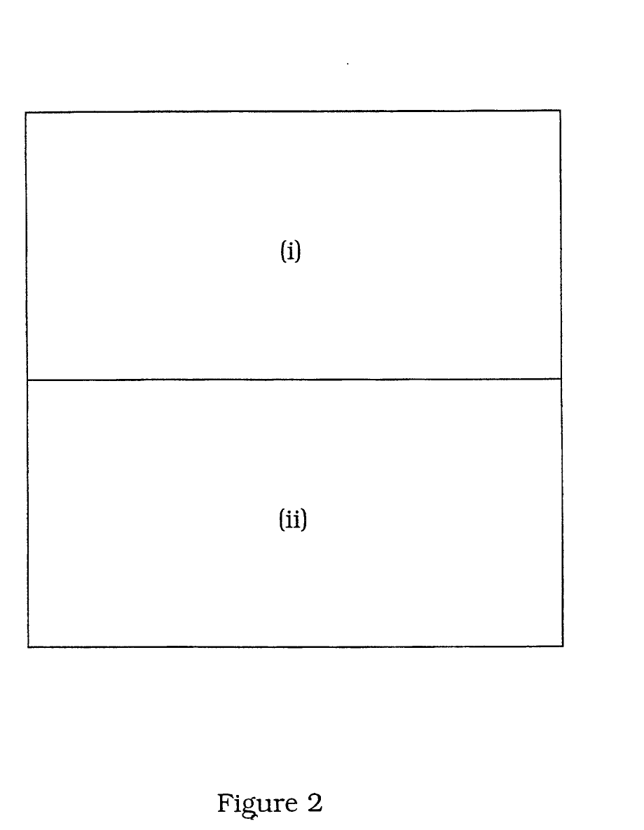

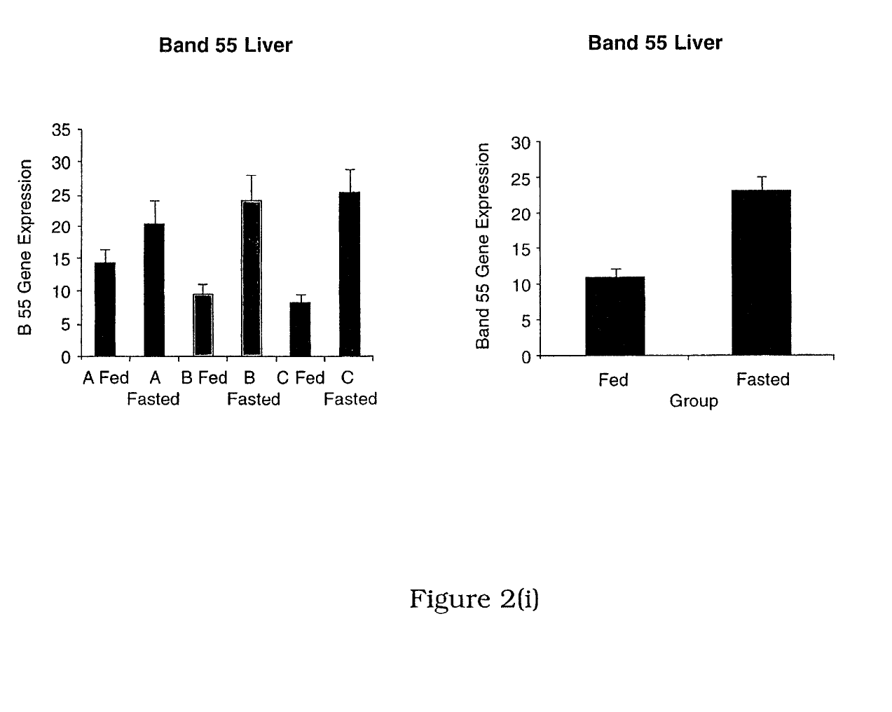

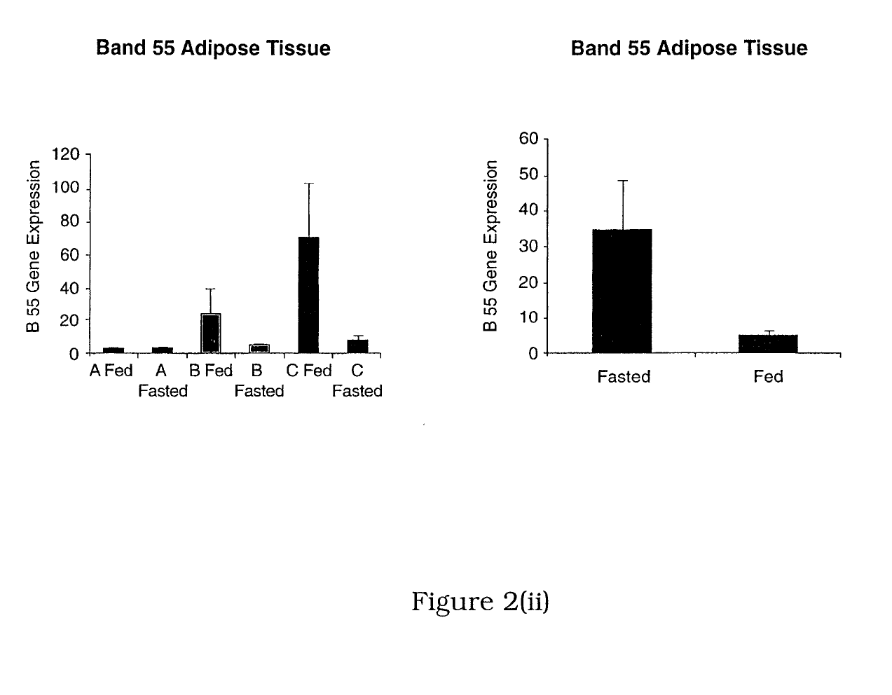

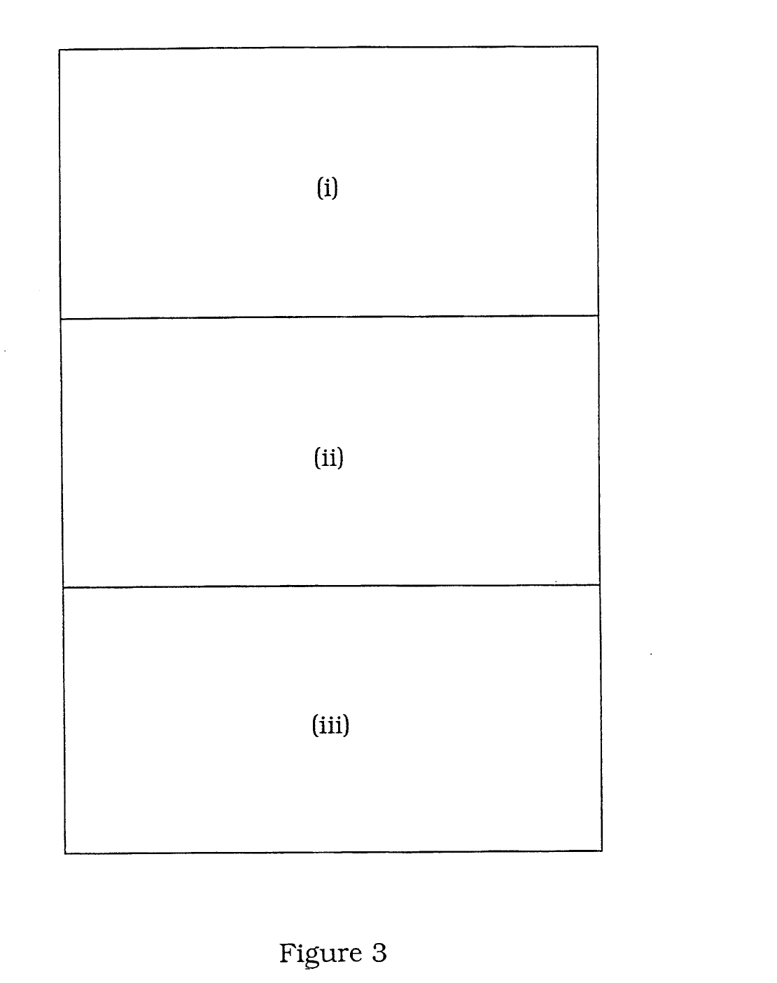

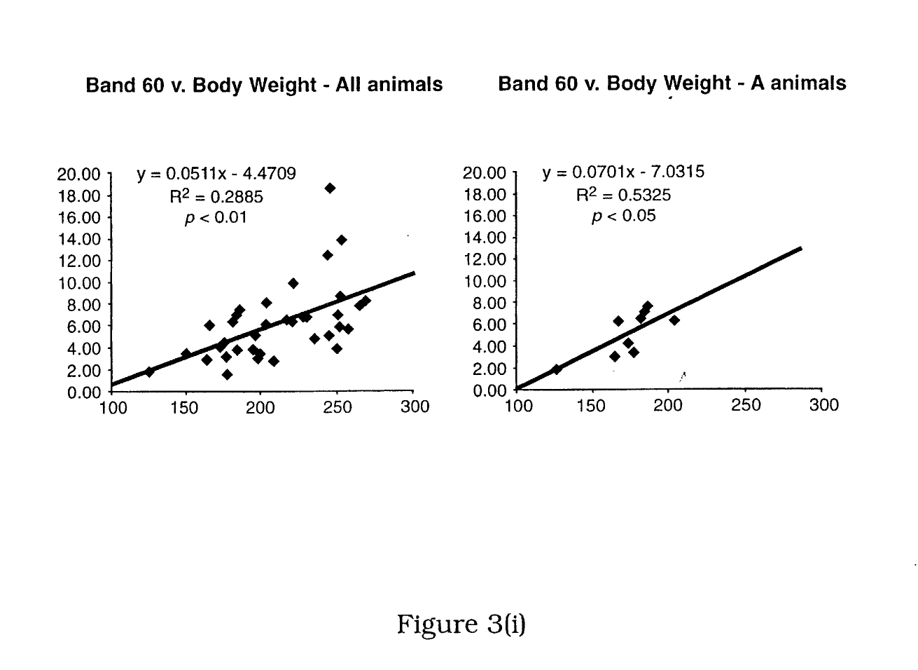

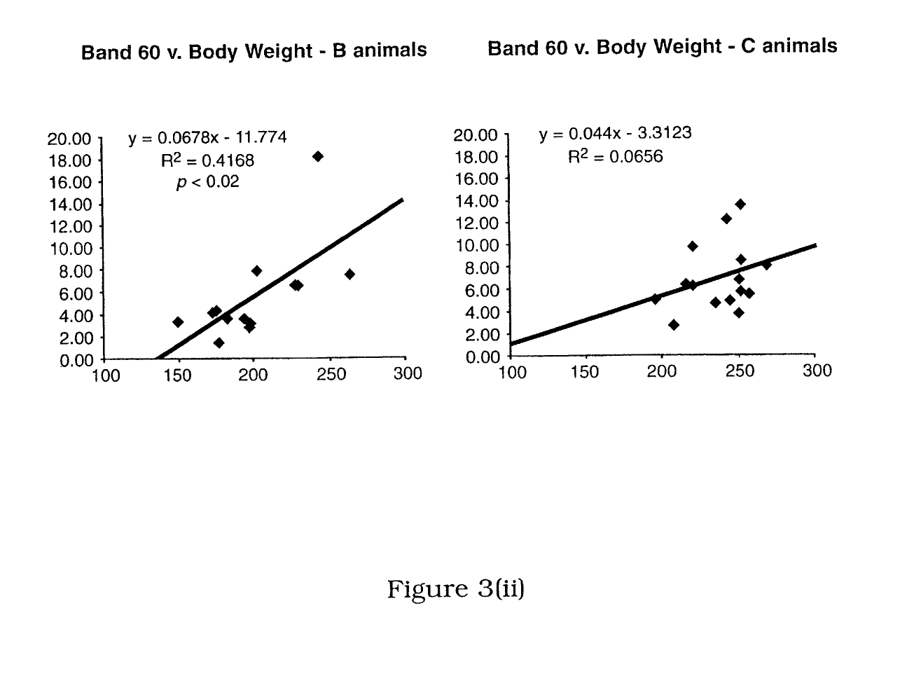

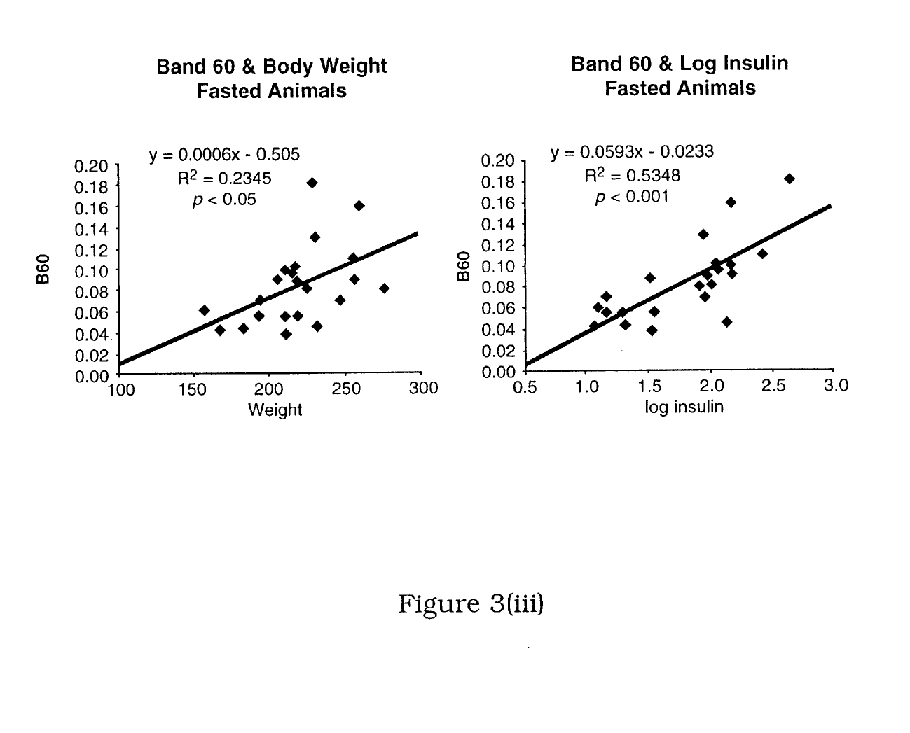

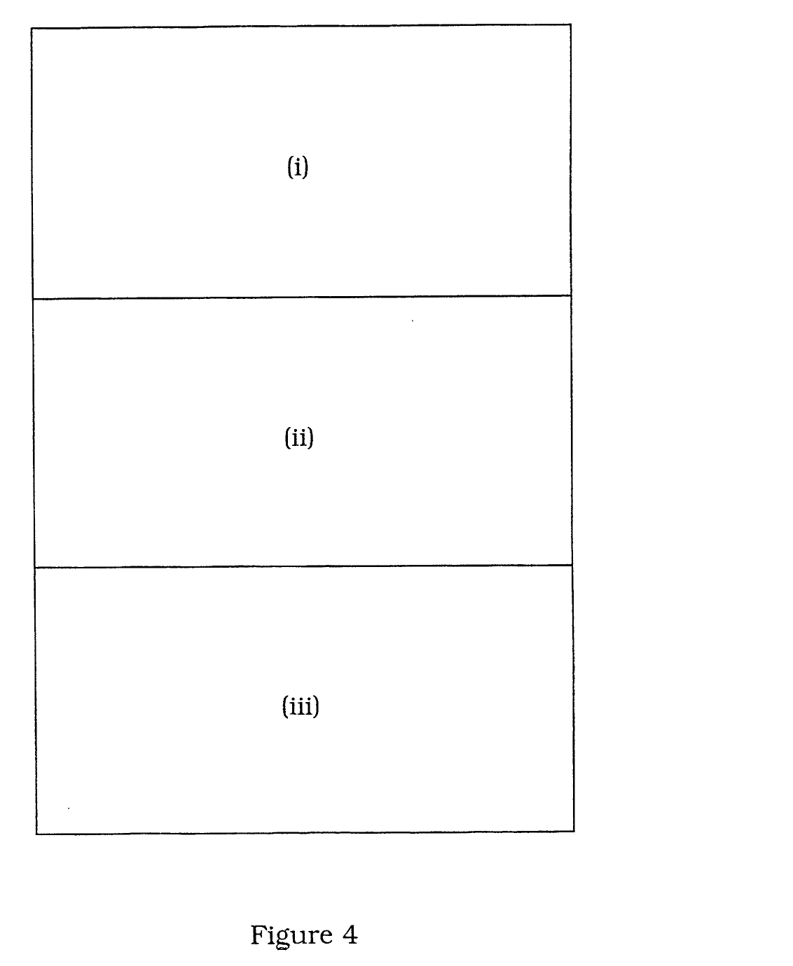

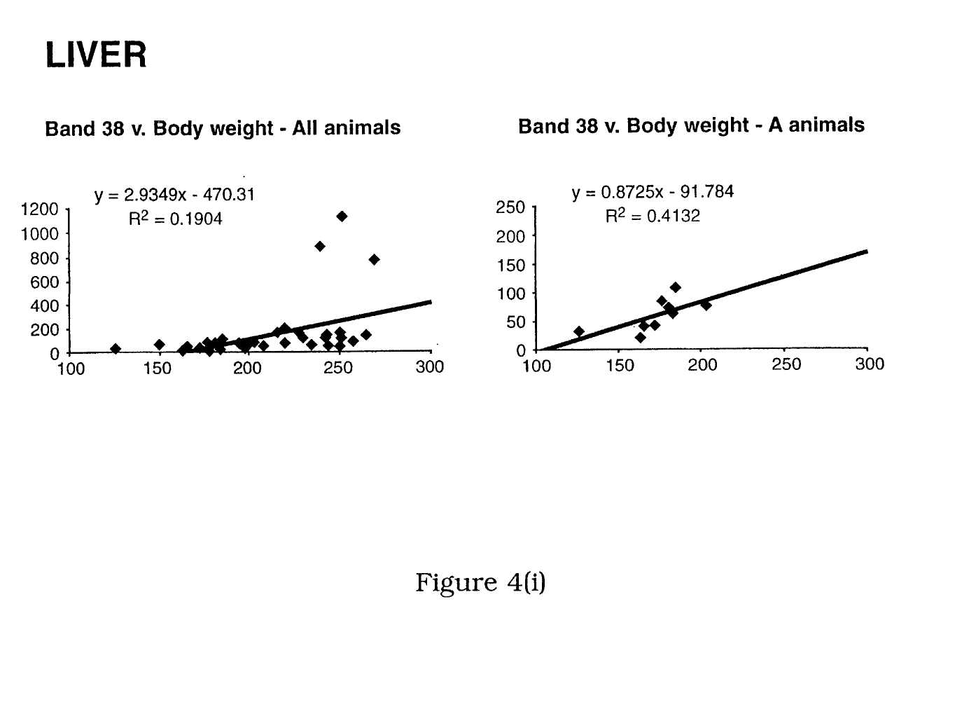

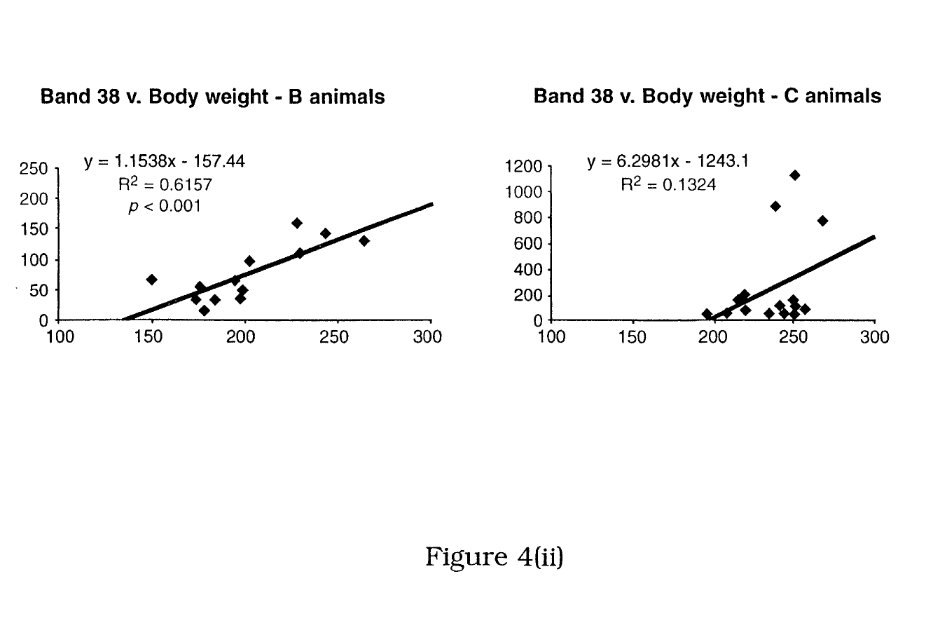

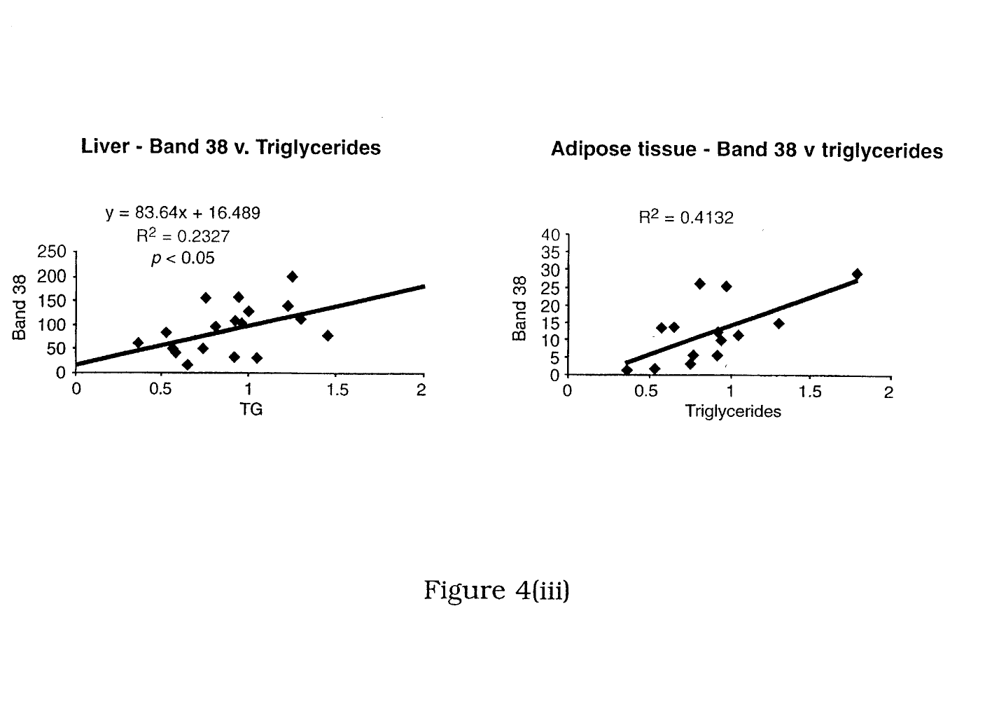

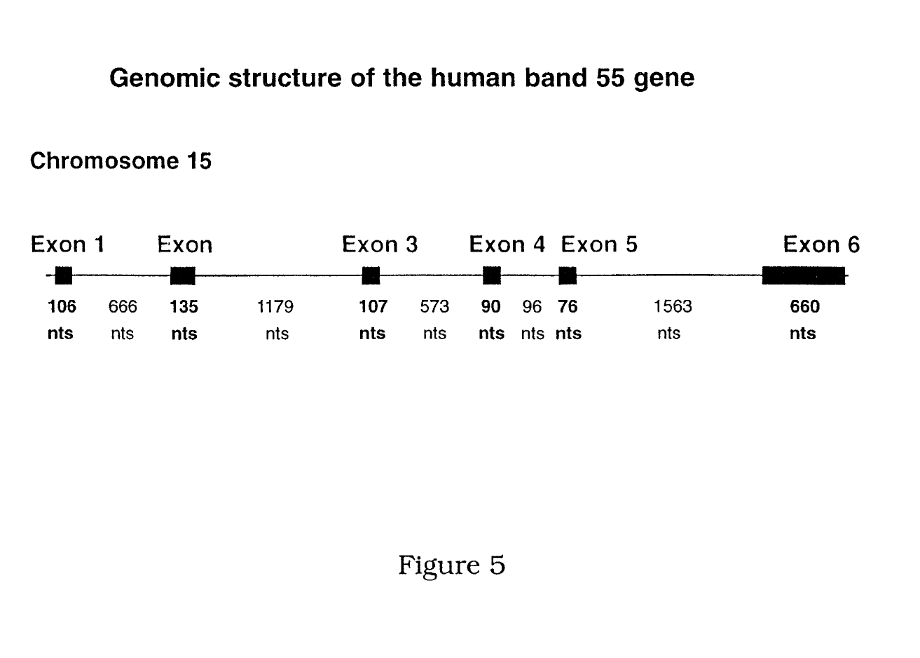

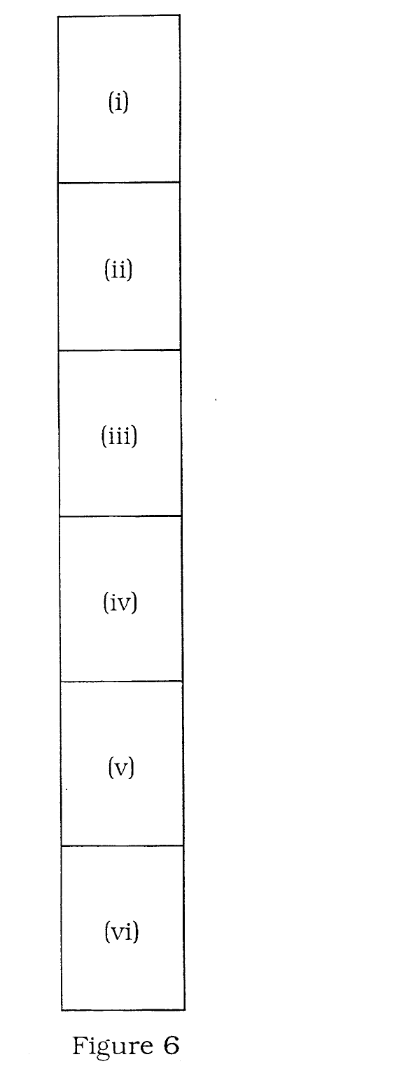

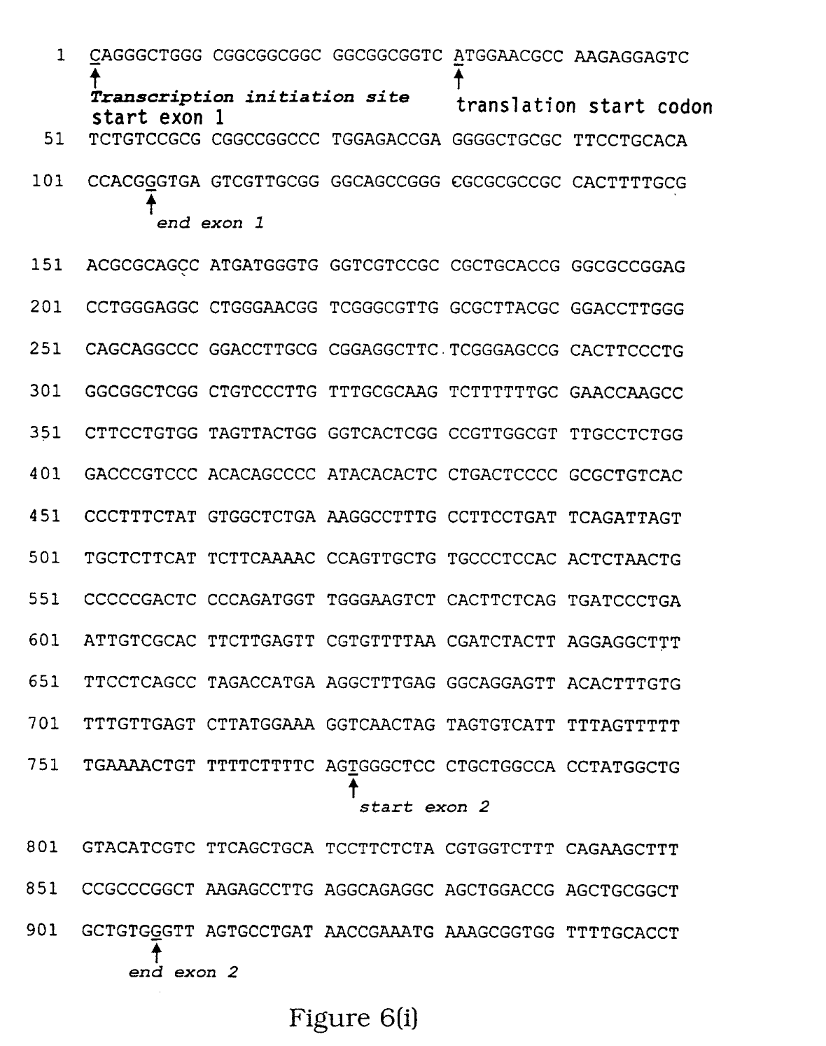

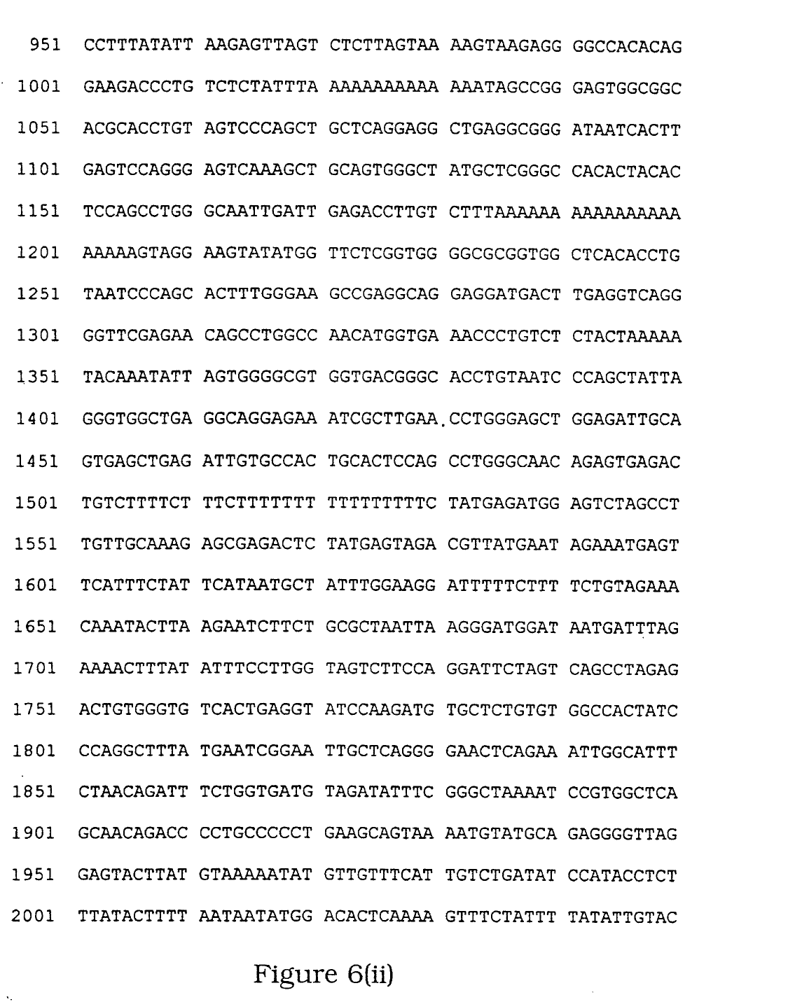

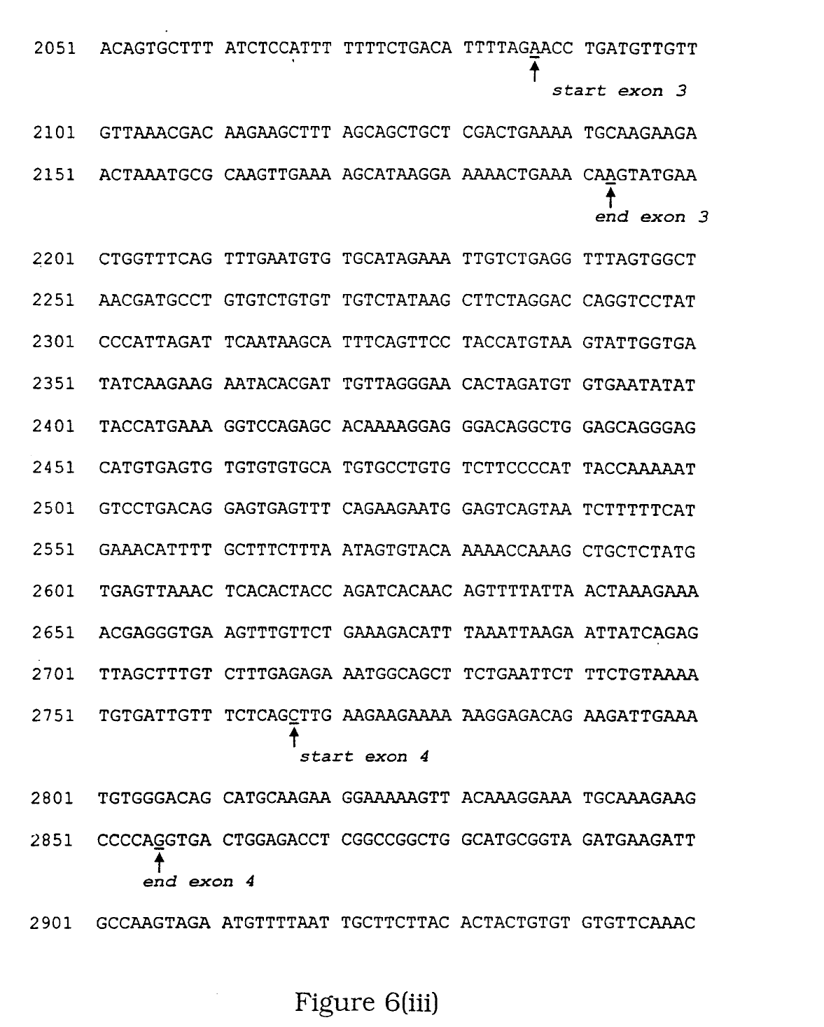

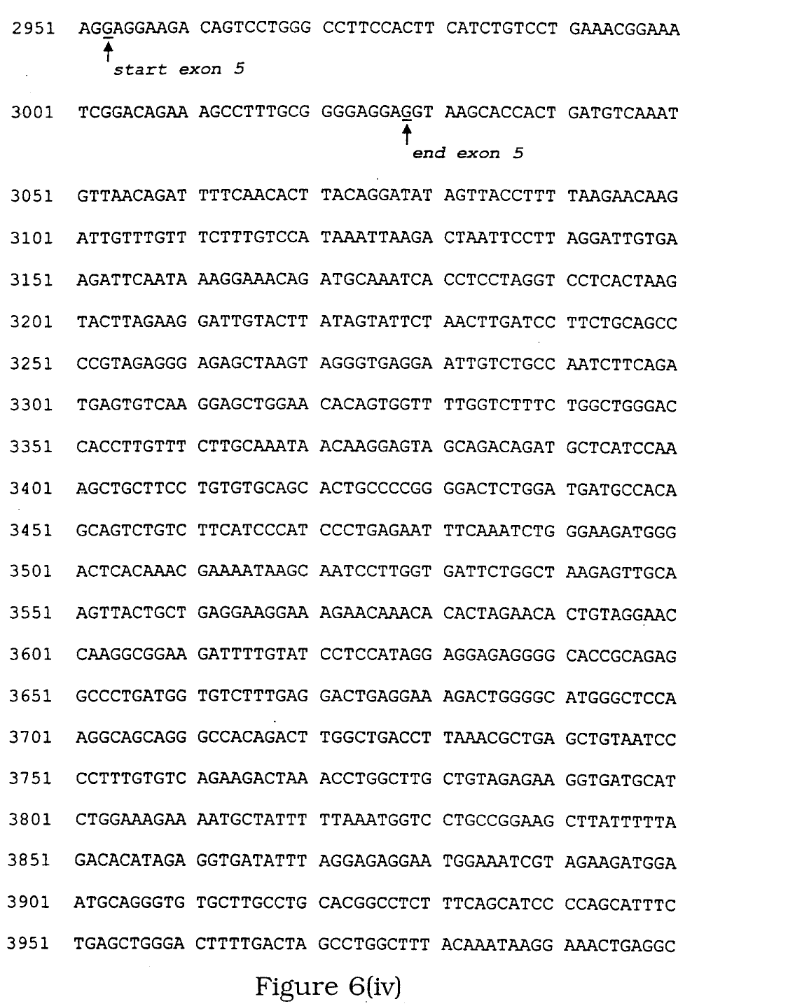

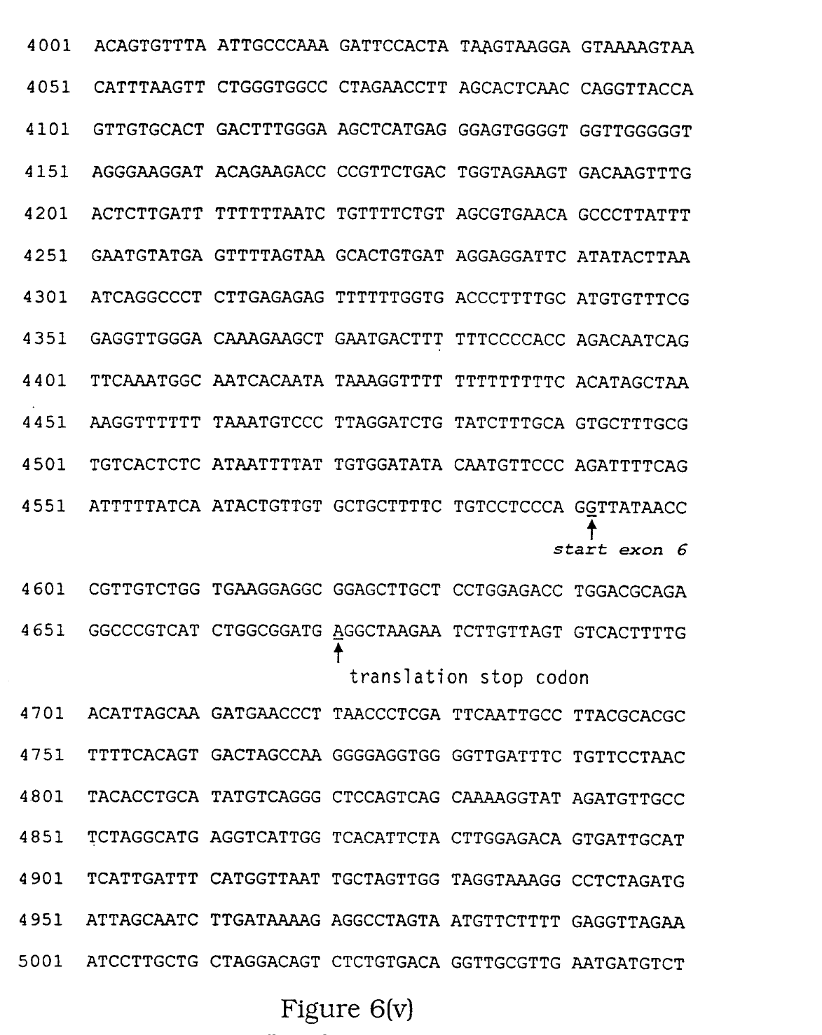

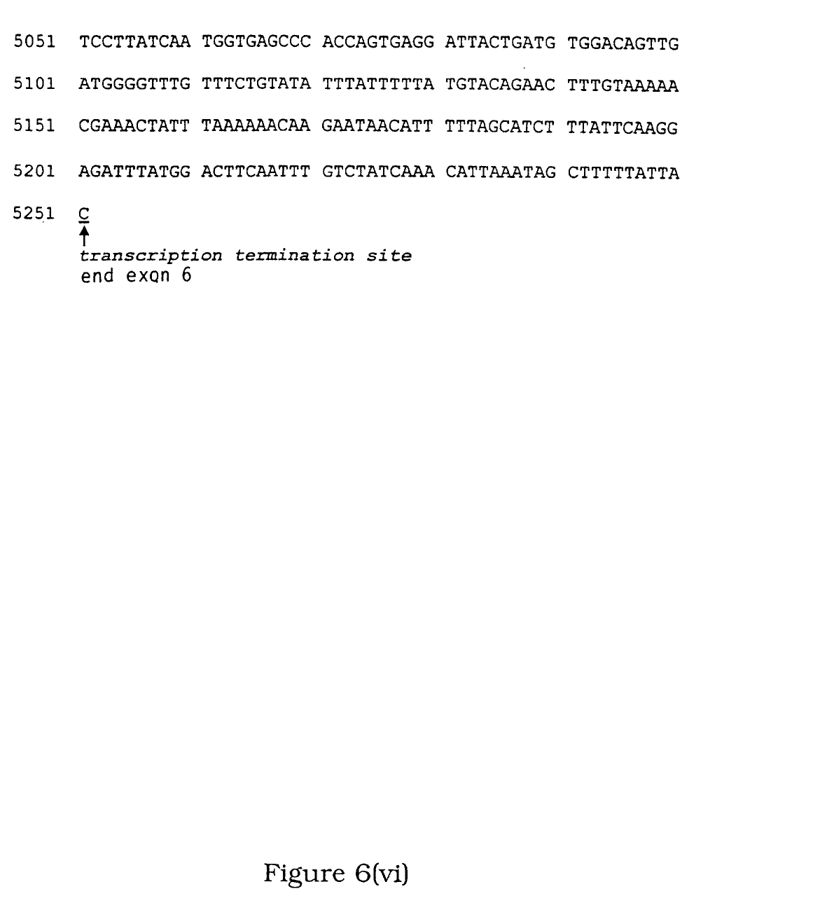

### DETAILED DESCRIPTION OF THE INVENTION

The present invention is predicated, in part, on the identification of novel genes which are differentially expressed in association with obesity, diabetes and energy metabolism.

Accordingly, one aspect of the present invention provides an isolated nucleic acid molecule or derivative, homologue or analogue thereof comprising a nucleotide sequence encoding or complementary to a sequence encoding a protein or a derivative, homologue or mimetic of said protein wherein said nucleic acid molecule is differentially expressed in liver tissue of obese animals compared to lean animals.

In another aspect, the present invention provides an isolated nucleic acid molecule or derivative, homologue or analogue thereof comprising a nucleotide sequence encoding or complementary to a sequence encoding a protein or a derivative, homologue or mimetic of said protein wherein said nucleic acid molecule is differentially expressed in liver tissue of fed animals compared to fasted animals.

The terms “lean” and “obese” are used in their most general sense but should be considered relative to the standard criteria for determining obesity. Generally, for human subjects the definition of obesity is BMI>30 (Risk Factor Prevalence 1990; Waters and Bennett, 1995).

The term “fasted” should be understood to mean that an animal is deprived of food. Preferably, the animal is fasted for at least 24 hours.

Conveniently, an animal model may be employed to study the physiology of obese and lean animals. In particular, the present invention is exemplified using the Psammomys obesus (the Israeli sand rat) an animal model of dietary-induced obesity and NIDDM. In its natural desert habitat, an active lifestyle and saltbush diet ensure that it remains lean and normoglycemic (Shafrir and Gutman, 1993). However, in a laboratory setting on a diet of ad libitum chow (on which many other animal species remain healthy), a range of pathophysiological responses are seen (Barnett et al, 1994a, b; Barnett et al, 1995). By the age of 16 weeks, more than half of the animals become obese and approximately one third develop NIDDM. Only hyperphagic animals go on to develop hyperglycemia, highlighting the importance of excessive energy intake in the pathophysiology of obesity and NIDDM in Psammomys obesus (Collier et al, 1997a; Walder et al, 1997a). Other phenotypes found include hyperinsulinemia, dyslipidemia, impaired glucose tolerance, cataracts and atherosclerosis (Collier et al, 1997a, b). Psammomys obesus exhibit a range of bodyweight and blood glucose and insulin levels which forms a continuous curve that closely resembles the patterns found in human populations, including the inverted U-shaped relationship between blood glucose and insulin levels known as “Starling's curve of the pancreas” (Barnett et al, 1994a; DeFronzo, 1988). It is the heterogeneity of the phenotypic response of Psammomys obesus which make it an ideal model to study the etiology and pathophysiology of obesity and NIDDM.

Another aspect of the present invention provides an isolated nucleic acid molecule or derivative, homologue or analogue thereof comprising a nucleotide sequence encoding, or a nucleotide sequence complementary to a nucleotide sequence encoding, an amino acid sequence substantially as set forth in <400>2 or a derivative, homologue or mimetic thereof or having at least about 45% similarity to at least 10 contiguous amino acids in <400>2.

The term “similarity” as used herein includes exact identity between compared sequences at the nucleotide or amino acid level. Where there is non-identity at the nucleotide level, “similarity” includes differences between sequences which result in different amino acids that are nevertheless related to each other at the structural, functional, biochemical and/or conformational levels. Where there is non-identity at the amino acid level, “similarity” includes amino acids that are nevertheless related to each other at the structural, functional, biochemical and/or conformational levels. The percentage similarity may be greater than 50% such as at least 70% or at least 80% or at least 90% or at least 95% or higher.

More particularly, the present invention contemplates an isolated nucleic acid molecule or derivative, homologue or analogue thereof comprising a nucleotide sequence substantially as set forth in <400>1 or a derivative or homologue thereof, or capable of hybridising to <400>1 under low stringency conditions.

Reference herein to a low stringency includes and encompasses from at least about 0% v/v to at least about 15% v/v formamide and from at least about 1M to at least about 2M salt for hybridisation, and at least about 1 M to at least about 2M salt for washing conditions. Alternative stringency conditions may be applied where necessary, such as medium stringency, which includes and encompasses from at least about 16% v/v to at least about 30% v/v formamide and from at least about 0.5M to at least about 0.9M salt for hybridisation, and at least about 0.5M to at least about 0.9M salt for washing conditions, or high stringency, which includes and encompasses from at least about 31% v/v to at least about 50% v/v formamide and from at least about 0.01M to at least about 0.15M salt for hybridisation, and at least about 0.01 M to at least about 0.15M salt for washing conditions. Stringency may be measured using a range of temperature such as from about 40° C. to about 65° C. Particularly useful stringency conditions are at 42° C. In general, washing is carried out at Tₘ=69.3+0.41 (G+C) % [19] =−12 [symbol] C. However, the Tₘ of a duplex DNA decreases by 1 [symbol] C with every increase of 1% in the number of mismatched based pairs (Bonner et al 1973).

Preferably, the present invention contemplates a nucleic acid molecule or a derivative, homologue or analogue thereof comprising a nucleotide sequence substantially as set forth in <400>1 or a derivative or homologue thereof or capable of hybridising to <400>1 under low stringency conditions and which encodes an amino acid sequence corresponding to an amino acid sequence set forth in <400>2 or a sequence having at least about 45% similarity to at least 10 contiguous amino acids in <400>2.

More particularly, the present invention contemplates a nucleic acid molecule comprising a sequence of nucleotides substantially as set forth in <400>1.

In another aspect the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>4 or a derivative, homologue or mimetic thereof or having at least about 45% similarity to at least 10 contiguous amino acids in <400>4.

More particularly, the present invention contemplates a nucleic acid molecule or derivative, homologue or analogue thereof comprising a nucleotide sequence substantially as set forth in <400>3 or a derivative or homologue thereof, or capable of hybridising to <400>3 under low stringency conditions.

Preferably, the present invention contemplates a nucleic acid molecule or derivative, homologue or analogue thereof comprising a nucleotide sequence substantially as set forth in <400>3 or a derivative or homologue thereof or capable of hybridising to <400>3 under low stringency conditions and which encodes an amino acid sequence corresponding to an amino acid sequence set forth in <400>4 or a sequence having at least about 45% similarity to at least 10 contiguous amino acids in <400>4.

More particularly, the present invention contemplates a nucleic acid molecule comprising a sequence of nucleotides substantially as set forth in <400>3.

In yet another aspect, the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>6 or a derivative, homologue or mimetic thereof or having at least about 45% similarity to at least 10 contiguous amino acids in <400>6.

More particularly, the present invention contemplates a nucleic acid molecule or derivative, homologue or analogue thereof comprising a nucleotide sequence substantially as set forth in <400>5 or a derivative or homologue thereof, or capable of hybridising to <400>5 under low stringency conditions.

Preferably, the present invention contemplates a nucleic acid molecule or derivative, homologue or analogue thereof comprising a nucleotide sequence substantially as set forth in <400>5 or a derivative, homologue or mimetic thereof or capable of hybridising to <400>5 under low stringency conditions and which encodes an amino acid sequence corresponding to an amino acid sequence set forth in <400>6 or a sequence having at least about 45% similarity to at least 10 contiguous amino acids in <400>6.

More particularly, the present invention contemplates a nucleic acid molecule comprising a sequence of nucleotides substantially as set forth in <400>5.

In yet another aspect, the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>8 or a derivative, homologue or mimetic thereof or having at least about 45% similarity to at least 10 contiguous amino acids in <400>8.

More particularly, the present invention contemplates a nucleic acid molecule or derivative, homologue or analogue thereof comprising a nucleotide sequence substantially as set forth in <400>7 or a derivative or homologue thereof, or capable of hybridising to <400>7 under low stringency conditions.

Preferably, the present invention contemplates a nucleic acid molecule or derivative, homologue or analogue thereof comprising a nucleotide sequence substantially as set forth in <400>7 or a derivative, homologue or mimetic thereof or capable of hybridising to <400>7 under low stringency conditions and which encodes an amino acid sequence corresponding to an amino acid sequence set forth in <400>8 or a sequence having at least about 45% similarity to at least 10 contiguous amino acids in <400>8.

More particularly, the present invention contemplates a nucleic acid molecule comprising a sequence of nucleotides substantially as set forth in <400>7.

In yet another aspect, the present invention provides a nucleic acid molecule or derivative, homologue or analogue thereof comprising a nucleotide sequence encoding, or a nucleotide sequence complementary to a nucleotide sequence encoding, an amino acid sequence substantially as set forth in <400>6 or a derivative, homologue or mimetic thereof or having at least about 45% similarity to at least 10 contiguous amino acids in <400>6.

More particularly, the present invention contemplates a nucleic acid molecule or derivative, homologue or analogue thereof comprising a nucleotide sequence substantially as set forth in <400>9 or a derivative or homologue thereof, or capable of hybridising to <400>9 under low stringency conditions.

Preferably, the present invention contemplates a nucleic acid molecule or derivative, homologue or analogue thereof comprising a nucleotide sequence substantially as set forth in <400>9 or a derivative, homologue or mimetic thereof or capable of hybridising to <400>9 under low stringency conditions and which encodes an amino acid sequence corresponding to an amino acid sequence set forth in <400>6 or a sequence having at least about 45% similarity to at least 10 contiguous amino acids in <400>6.

More particularly, the present invention contemplates a nucleic acid molecule comprising a sequence of nucleotides substantially as set forth in <400>9.

The nucleic acid molecules according to these aspects of the present invention correspond herein to B38, B55 and B60. The expression pattern of these genes has been determined, inter alia, to indicate an involvement in the regulation of one or more of obesity, diabetes and/or energy metabolism. In addition to the differential expression of B38, B55 and B60 in the liver tissue of lean vs obese animals and fed vs fasted animals these genes are also expressed in other tissues including, but in no way limited to, muscle and hypothalamus. Reference to “B38, B55 and B60” in italised text should be understood as a reference to the nucleic acid molecule while reference to “B38, B55 and B60” in non-italised text should be understood as a reference to the expression product. Murine B38 comprises the amino acid sequence set forth in <400>2 and and the cDNA sequence set forth in <400>1. Murine B55 comprises the amino acid sequence set forth in <400>4 and the cDNA sequence set forth in <400>3. Human B55 comprises the amino acid sequence set forth in <400>6 and the cDNA sequence set forth in <400>5. The genomic sequence of human B55 is provided in <400>9. Murine B60 comprises the amino acid sequence set forth in <400>8 and the cDNA sequence set forth in <400>7. The nucleic acid molecle encoding B38, B55 or B60 is preferably a sequence of deoxyribonucleic acids such as a cDNA sequence or a genomic sequence. A genomic sequence may also comprise exons and introns. A genomic sequence may also include a promoter region or other regulatory regions. It should be understood that the genomic sequence disclosed herein in <400>9 corresponds only to that part of the sequence running from the transcription initiation site to the transcription termination site. Accordingly, the <400>9 sequence and other genomic sequences encompassed by the present invention may comprise either more or less sequence than that encompassed from the transcription initiation site to the transcription termination site. For example, it may comprise additional nontranslated sequences such as regulatory sequences located up- or down-stream of the transcription start/stop sites.

Another aspect of the present invention contemplates a genomic nucleic acid molecule or derivative homologue or analogue thereof capable of hybridising to <400>1 or a derivative or homologue thereof under low stringency conditions at 42° C.

Yet another aspect of the present invention contemplates a genomic nucleic acid molecule or derivative homologue or analogue thereof capable of hybridising to <400>3 or a derivative or homologue thereof under low stringency conditions at 42° C.

Still another aspect of the present invention contemplates a genomic nucleic acid molecule or derivative homologue or analogue thereof capable of hybridising to <400>7 or a derivative or homologue thereof under low stringency conditions at 42° C.

Still yet another aspect of the present invention contemplates a genomic nucleic acid molecule or derivative homologue or analogue thereof capable of hybridising to <400>5 or a derivative or homologue thereof under low stringency conditions.

Yet another aspect of the present invention contemplates a cDNA nucleic acid molecule or derivative, homologue or analogue thereof capable of hybridising to <400>9 or a derivative or homologue thereof under low stringency conditions.

Reference herein to “B38, B55, B60” and “B38, B55, B60” should be understood as a reference to all forms of these molecules and derivatives, homologues, analogues, chemical equivalents and mimetics thereof including, for example, any peptide and cDNA isoforms which arise from alternative splicing of B38, B55 or B60 mRNA or mutants or polymorphic variants of B38, B55, B60 or B38, B55, B60.

The molecules disclosed herein have been isolated from the Israeli sand rat. However, it should be understood that the protein and/or gene molecules may also be isolated from any other human or non-human species.

Derivatives include fragments, parts, portions, mutants, variants and mimetics from natural, synthetic or recombinant sources including fusion proteins. Parts or fragments include, for example, active regions of B38, B55 or B60. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Chemical and functional equivalents of B38, B55, B60 or B38, B55, B60 should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 1.

TABLE 1

| Non-conventional | | | |
|---|---|---|---|
| amino acidamino acid | Code | Non-conventional | Code |
| α-aminobutyric acid | Abu | L-N-methylalanine | Nmala |
| α-amino-α-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbornyl- | Norb | L-N-methylglutamine | Nmgln |
| carboxylate | | L-N-methylglutamic acid | Nmglu |
| cyclohexylalanine | Chexa | L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucin | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | a-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenyl | Mhphe |
| | | alanine | |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenyl | Nmhphe |
| | | alanine | |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl-Nmbc | | | |
| ethylamino)cyclopropane | | | |

Crosslinkers can be used, for example, to stabilise 3D conformations, using homo-bifunctional crosslinkers such as the bifunctional imido esters having (CH2)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

The nucleic acid molecule of the present invention is preferably in isolated form or ligated to a vector, such as an expression vector. By “isolated” is meant a nucleic acid molecule having undergone at least one purification step and this is conveniently defined, for example, by a composition comprising at least about 10% subject nucleic acid molecule, preferably at least about 20%, more preferably at least about 30%, still more preferably at least about 40-50%, even still more preferably at least about 60-70%, yet even still more preferably 80-90% or greater of subject nucleic acid molecule relative to other components as determined by molecular weight, encoding activity, nucleotide sequence, base composition or other convenient means. The nucleic acid molecule of the present invention may also be considered, in a preferred embodiment, to be biologically pure.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

In a particularly preferred embodiment, the nucleotide sequence corresponding to B38 is a cDNA sequence comprising a sequence of nucleotides as set forth in <400>1 or a derivative, homologue or analogue thereof including a nucleotide sequence having similarity to <400>1.

In another particularly preferred embodiment, the nucleotide sequence corresponding to B55 is a cDNA sequence comprising a sequence of nucleotides as set forth in <400>3 or a derivative, homologue or analogue thereof including a nucleotide sequence having similarity to <400>3.

In still another particularly preferred embodiment, the nucleotide sequence corresponding to B55 is a cDNA sequence comprising a sequence of nucleotides as set forth in <400>5 or a derivative homologue or analogue thereof including a nucleotide sequence having similarity to <400>5.

In yet another preferred embodiment, the nucleotide sequence corresponding to B55 is a genomic sequence comprising a sequence of nucleotides as set forth in <400>9 or a derivative homologue or analogue thereof including a nucleotide sequence having similarity to <400>9.

In yet another particularly preferred embodiment, the nucleotide sequence corresponding to B60 is a cDNA sequence comprising a sequence of nucleotides as set forth in <400>7 or a derivative, homologue or analogue thereof including a nucleotide sequence having similarity to <400>7.

A derivative of the nucleic acid molecule of the present invention also includes a nucleic acid molecule capable of hybridising to a nucleotide sequence as set forth in any one or more of <400>1, >400>3, >400>5, >400>7 or <400>9 under low stringency conditions. Preferably, low stringency is at 42° C.

The nucleic acid molecule may be ligated to an expression vector capable of expression in a prokaryotic cell (e.g. E. coli) or a eukaryotic cell (e.g. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of sphingosine kinase which forms are encompassed by the present invention.

The present invention extends to the expression product of the nucleic acid molecules as hereinbefore defined.

Expression products are B38, B55 and B60 having an amino acid sequence as set forth in <400>2, >400>4, >400>6 and <400>8, respectively, or are derivatives, analogues, homologues, chemical equivalents or mimetics thereof as defined above or are derivatives or mimetics having an amino acid sequence of at least about 45% similarity to at least 10 contiguous amino acids in the amino acid sequence set forth in <400>2, <400>4, >400>6 and <400>8, respectively, or a derivative or mimetic thereof.

Another aspect of the present invention is directed to an isolated protein selected from the list consisting of:

(i) a protein encoded by a novel nucleic acid molecule which molecule is differentially expressed in liver tissue of obese animals compared to lean animals or a derivative, homologue, analogue, chemical equivalent or mimetic thereof.

(ii) a protein encoded by a novel nucleic acid molecule which molecule is differentially expressed in liver tissue of fed animals compared to fasted animals or a derivative, homologue, analogue, chemical equivalent or mimetic thereof.

(iii) B38, B55 or B60 or a derivative, homologue, analogue, chemical equivalent or mimetic thereof.

(iv) a protein having an amino acid sequence substantially as set forth in <400>2 or a derivative, homologue or mimetic thereof or a sequence having at least about 45% similarity to at least 1 0 contiguous amino acids in <400>2 or a derivative, homologue, analogue, chemical equivalent or mimetic of said protein.

(v) a protein having an amino acid sequence substantially as set forth in <400>4 or a derivative, homologue or mimetic thereof or a sequence having at least about 45% similarity to at least 10 contiguous amino acids in <400>4 or a derivative, homologue, analogue, chemical equivalent or mimetic of said protein.

(vi) a protein having an amino acid sequence substantially as set forth in <400>6 or a derivative, homologue or mimetic thereof or a sequence having at least about 45% similarity to at least 10 contiguous amino acids in <400>6 or a derivative, homologue, analogue, chemical equivalent or mimetic of said protein.

(vii) a protein having an amino acid sequence substantially as set forth in <400>8 or a derivative, homologue or mimetic thereof or a sequence having at least about 45% similarity to at least 10 contiguous amino acids in <400>8 or a derivative, homologue, analogue, chemical equivalent or mimetic of said protein.

(viii) a protein encoded by a nucleotide sequence substantially as set forth in <400>1 or a derivative, homologue or analogue thereof or a sequence encoding an amino acid sequence having at least about 45% similarity to at least 10 contiguous amino acids in <400>2 or a derivative, homologue, analogue, chemical equivalent or mimetic of said protein.

(ix) a protein encoded by a nucleotide sequence substantially as set forth in <400>3 or a derivative, homologue or analogue thereof or a sequence encoding an amino acid sequence having at least about 45% similarity to at least 10 contiguous amino acids in <400>4 or a derivative, homologue, analogue, chemical equivalent or mimetic of said protein.

(x) a protein encoded by a nucleotide sequence substantially as set forth in <400>5 or a derivative, homologue or analogue thereof or a sequence encoding an amino acid sequence having at least about 45% similarity to at least 10 contiguous amino acids in <400>6 or a derivative, homologue, analogue, chemical equivalent or mimetic of said protein.

(xi) a protein encoded by a nucleotide sequence substantially as set forth in <400>7 or a derivative, homologue or analogue thereof or a sequence encoding an amino acid sequence having at least about 45% similarity to at least 10 contiguous amino acids in <400>8 or a derivative, homologue, analogue, chemical equivalent or mimetic of said protein.

(xii) a protein encoded by a nucleotide sequence substantially as set forth in <400>9 or a derivative, homologue or analogue thereof or a sequence encoding an amino acid sequence having at least about 45% similarity to at least 10 contiguous amino acids in <400>6 or a derivative, homologue, analogue, chemical equivalent or mimetic of said protein.

(xiii) a protein encoded by a nucleic acid molecule capable of hybridising to the nucleotide sequence as set forth in <400>1 or a derivative, homologue or analogue thereof under low stringency conditions and which encodes an amino acid sequence substantially as set forth in <400>2 or a derivative, homologue or mimetic thereof or an amino acid sequence having at least about 45% similarity to at least 10 contiguous amino acids in <400>2.

(xiv) a protein encoded by a nucleic acid molecule capable of hybridising to the nucleotide sequence as set forth in <400>3 or a derivative, homologue or analogue thereof under low stringency conditions and which encodes an amino acid sequence substantially as set forth in <400>4 or a derivative, homologue or mimetic thereof or an amino acid sequence having at least about 45% similarity to at least 10 contiguous amino acids in <400>4.

(xv) a protein encoded by a nucleic acid molecule capable of hybridising to the nucleotide sequence as set forth in <400>5 or a derivative, homologue or analogue thereof under low stringency conditions and which encodes an amino acid sequence substantially as set forth in <400>6 or a derivative, homologue or mimetic thereof or an amino acid sequence having at least about 45% similarity to at least 10 contiguous amino acids in <400>6.

(xvi) a protein encoded by a nucleic acid molecule capable of hybridising to the nucleotide sequence as set forth in <400>7 or a derivative, homologue or analogue thereof under low stringency conditions and which encodes an amino acid sequence substantially as set forth in <400>8 or a derivative, homologue or mimetic thereof or an amino acid sequence having at least about 45% similarity to at least 10 contiguous amino acids in <400>8.

(xvii) a protein encoded by a nucleic acid molecule capable of hybridising to the nucleotide sequence as set forth in <400>9 or a derivative, homologue or analogue thereof under low stringency conditions and which encodes an amino acid sequence substantially as set forth in <400>6 or a derivative, homologue or mimetic thereof or an amino acid sequence having at least about 45% similarity to at least 10 contiguous amino acids in <400>6.

(xviii) a protein as defined in any one of paragraphs (i) to (xvii) in a homodimeric form.

(xix) a protein as defined in any one of paragraphs (i) to (xvii) in a heterodimeric form.

The protein of the present invention is preferably in isolated form. By “isolated” is meant a protein having undergone at least one purification step and this is conveniently defined, for example, by a composition comprising at least about 10% subject protein, preferably at least about 20%, more preferably at least about 30%, still more preferably at least about 40-50%, even still more preferably at least about 60-70%, yet even still more preferably 80-90% or greater of subject protein relative to other components as determined by molecular weight, amino acid sequence or other convenient means. The protein of the present invention may also be considered, in a preferred embodiment, to be biologically pure.

Without limiting the theory or mode of action of the present invention, the expression of B38 is thought to relate to body weight and circulating triglycerides. Modulation of B38 expression is thought, inter alia, to regulate energy balance via effects on energy intake and also effects on carbohydrate/fat metabolism. The energy intake effects are likely to be mediated via the central nervous system but peripheral effects on the metabolism of both carbohydrate and fat are possible. The expression of B55 is thought to be regulated by fasting and feeding, accordingly, regulating the expression and/or activity of this gene or its expression product could provide a mechanism for regulating both body weight and energy metabolism, including carbohydrate and fat metabolism. Since B55 is differentially regulated in diabetes, it is also thought to provide a diabetic target. Finally, B60 gene expression has been shown to associate with body weight. In this regard, B60 is thought to exhibit similar effects to B38. To the extent that it is not specified, reference to B38, B55, B60 or B38, B55, B60 includes reference to derivatives, homologs, analogs, chemical equivalents and mimetics thereof. For example, reference to B38 and chemical equivalents thereof should be understood to encompass the complement components C3a and C5a which comprise a region of high homology with B38. These molecules comprise an anaphylatoxin-like domain and have been shown to increase hepatic glucose output.

Accordingly, regulating the functional activity and/or levels of these molecules provides a mechanism for the therapeutic and prophylactic treatment of conditions such as obesity, anorexia, energy imbalance and diabetes. The cloning and sequencing of these genes and expression products now provides further molecules for use in such treatments. These molecules may also be useful in the agricultural industry to assist in the generation of leaner animals, or where required, of obese animals. Accordingly, the mammal contemplated by the present invention includes, but is not limited to, humans, primates, livestock animals (e.g. pigs, sheep, cows, horses, donkeys), laboratory test animals (e.g. mice, rats, guinea pigs, hamsters, rabbits), companion animals (e.g. dogs, cats) and captured wild animals (e.g. foxes, kangaroos and deer). A particularly preferred mammal is a human, primate or livestock animal.

Accordingly, the present invention contemplates therapeutic and prophylactic uses of 3B38, B55 and B60 amino acid and nucleic acid molecules, in addition to B38, B55 and B60 agonistic and antagonistic agents.

The present invention contemplates, therefore, a method of modulating expression of B38, B55 and/or B60 in a mammal, said method comprising contacting the B38, B55 and/or B60 gene with an effective amount of an agent for a time and under conditions sufficient to upregulate, downregulate or otherwise modulate expression of B38, B55 and/or B60. For example, antisense sequences such as oligonucleotides may be utilised.

Conversely, nucleic acid molecules encoding B38, B55 and/or B60 or derivatives thereof may be introduced to upregulate one or more specific functional activities.

Another aspect of the present invention contemplates a method of modulating activity of B38, B55 and/or B60 in a subject, said method comprising administering to said subject a modulating effective amount of an agent for a time and under conditions sufficient to increase or decrease B38, B55 and/or B60 activity.

Modulation of said activity by the administration of an agent to a mammal can be achieved by one of several techniques, including but in no way limited to introducing into said mammal a proteinaceous or non-proteinaceous molecule which:

(i) modulates expression of B38, B55 and/or B60;

(ii) functions as an antagonist of B38, B55 and/or B60;

(iii) functions as an agonist of B38, B55 and/or B60.

Said proteinaceous molecule may be derived from natural or recombinant sources including fusion proteins or following, for example, natural product screening. Said non-proteinaceous molecule may be, for example, a nucleic acid molecule or may be derived from natural sources, such as for example natural product screening or may be chemically synthesised. The present invention contemplates chemical analogues of B38, B55 and/or B60 or small molecules capable of acting as agonists or antagonists. Chemical agonists may not necessarily be derived from B38, B55 and/or B60 but may share certain conformational similarities. Alternatively, chemical agonists may be specifically designed to mimic certain physiochemical properties. Antagonists may be any compound capable of blocking, inhibiting or otherwise preventing B38, B55 and/or B60 from carrying out their normal biological functions. Antagonists include monoclonal antibodies and antisense nucleic acids which prevent transcription or translation of B38, B55 and/or B60 genes or mRNA in mammalian cells. Modulation of expression may also be achieved utilising antigens, RNA, ribosomes, DNAzymes, RNA aptamers or antibodies.

Said proteinaceous or non-proteinaceous molecule may act either directly or indirectly to modulate the expression of B38, B55 and/or B60 or the activity of B38, B55 and/or B60.

Said molecule acts directly if it associates with B38, B55 and/or B60 or B38, B55 and/or B60 to modulate expression or activity. Said molecule acts indirectly if it associates with a molecule other than B38, B55 and/or B60 or B38, B55 and/or B60 which other molecule either directly or indirectly modulates the expression or activity of B38, B55 and/or B60 or B38, B55 and/or B60. Accordingly, the method of the present invention encompasses the regulation of B38, B55 and/or B60 or B38, B55 and/or B60 expression or activity via the induction of a cascade of regulatory steps.

The molecules which may be administered to a mammal in accordance with the present invention may also be linked to a targeting means such as a monoclonal antibody, which provides specific delivery of these molecules to the target cells.

A further aspect of the present invention relates to the use of the invention in relation to mammalian disease conditions. For example, the present invention is particularly useful, but in no way limited to, use in a therapeutic or prophylactic treatment of obesity, anorexia, diabetes or energy imbalance.

Accordingly, another aspect of the present invention relates to a method of treating a mammal suffering from a condition characterised by one or more symptoms of obesity, anorexia, diabetes and/or energy imbalance said method comprising administering to said mammal an effective amount of an agent for a time and under conditions sufficient to modulate the expression of B38, B55 and/or B60 or sufficient to modulate the activity of B38, B55 and/or B60.

In another aspect the present invention relates to a method of treating a mammal suffering from a disease condition characterised by one or more symptoms of obesity, anorexia, diabetes or energy imbalance said method comprising administering to said mammal an effective amount of B38, B55 and/or B60 or B38, B55 and/or B60.

An [symbol] effective amount [symbol] means an amount necessary at least partly to attain the desired immune response, or to delay the onset or inhibit progression or halt altogether, the onset or progression of a particular condition of the individual to be treated, the taxonomic group of the individual to be treated, the degree of protection desired, the formulation of the vaccine, the assessment of the medical situation, and other relevant factors. It is expected that the amount will fall in a relatively broad range that can be determined through routine trials.

In accordance with these methods, B38, B55 and/or B60 or B38, B55 and/or B60 or agents capable of modulating the expression or activity of said molecules may be coadministered with one or more other compounds or other molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect the present invention relates to the use of an agent capable of modulating the expression of B38, B55 and/or B60 or a derivative, homologue or analogue thereof in the manufacture of a medicament for the treatment of a condition characterised by obesity, anorexia, diabetes and/or energy imbalance.

In still yet another aspect the present invention relates to the use of an agent capable of modulating the activity of B38, B55 and/or B60 or a derivative, homologue, analogue, chemical equivalent or mimetic thereof in the manufacture of a medicament for the treatment of a condition characterised by obesity, anorexia, diabetes and/or energy imbalance.

A further aspect of the present invention relates to the use of B38, B55 and/or B60 or derivative, homologue or analogue thereof or B38, B55 and/or B60 or derivative, homologue, analogue, chemical equivalent or mimetic thereof in the manufacture of a medicament for the treatment of a condition characterised by obesity, anorexia, diabetes and/or energy imbalance.

Still yet another aspect of the present invention relates to agents for use in modulating the expression of B38, B55 and/or B60 or a derivative, homologue or analogue thereof.

A further aspect relates to agents for use in modulating B38, B55 and/or B60 activity or a derivative, homologue, analogue, chemical equivalent or mimetic thereof.

Still another aspect of the present invention relates to B38, B55 and/or B60 or derivative, homologue or analogue thereof or B38, B55 and/or B60 or derivative, homologue, analogue, chemical equivalent or mimetic thereof for use in treating a condition characterised by one or more symptoms of obesity, anorexia, diabetes and/or energy imbalance.

In a related aspect of the present invention, the mammal undergoing treatment may be a human or an animal in need of therapeutic or prophylactic treatment.

In another aspect, the present invention contemplates a pharmaceutical composition comprising a modulator of B38, B55 and/or B60 expression or B38, B55 and/or B60 activity and one or more pharmaceutically acceptable carriers and/or diluents. In another embodiment, the pharmaceutical composition comprises B38, B55 and/or B60 or B38, B55 and/or B60 or a derivative, homologue, analogue, chemical equivalent or mimetic thereof and one or more pharmaceutically acceptable carriers and/or diluents. For brevity, all such components of such a composition are referred to as “active components”.

The compositions of active components in a form suitable for injectable use include sterile aqueous solutions (where water soluble) and sterile powders for the extemporaneous preparation of sterile injectable solutions. In all cases, the form must be sterile and must be fluid to the extent that easy syringability exists. It must be stable under the conditions of manufacture and storage and must be preserved against the contaminating action of microorganisms such as bacteria and fungi.

The carrier can be a solvent or other medium containing, for example, water, ethanol, polyol (for example, glycerol, propylene glycol and liquid polyethylene glycol, and the like), suitable mixtures thereof, and vegetable oils.

The preventions of the action of microorganisms can be brought about by various antibacterial and antifungal agents, for example, parabens, chlorobutanol, phenol, sorbic acid, thirmerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active components in the required amount in the appropriate solvent with optionally other ingredients, as required, followed by sterilization by, for example, filter sterilization, irradiation or other convenient means. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active components are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions is such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the following: A binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such a sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Pharmaceutically acceptable carriers and/or diluents include any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active material calculated to produce the desired therapeutic effect in association with the required pharmaceutical carrier. The specification for the novel dosage unit forms of the invention are dictated by and directly dependent on (a) the unique characteristics of the active material and the particular therapeutic effect to be achieved, and (b) the limitations inherent in the art of compounding such an active material for the treatment of disease in living subjects having a diseased condition in which bodily health is impaired as herein disclosed in detail.

The principal active component may be compounded for convenient and effective administration in sufficient amounts with a suitable pharmaceutically acceptable carrier in dosage unit form. A unit dosage form can, for example, contain the principal active component in amounts ranging from 0.5 μg to about 2000 mg. Expressed in proportions, the active compound is generally present in from about 0.5 μg to about 2000 mg/ml of carrier. In the case of compositions containing supplementary active ingredients, the dosages are determined by reference to the usual dose and manner of administration of the said ingredients.

In general terms, effective amounts will range from 0.01 ng/kg/body weight to above 10,000 mg/kg/body weight. Alternative amounts range from 0.1 ng/kg/body weight to above 1000 mg/kg/body weight.

The pharmaceutical composition may also comprise genetic molecules such as a vector capable of transfecting target cells where the vector carries a nucleic acid molecule capable of expressing the active ingredients or modulating the expression of the active ingredients or activity. The vector may, for example, be a viral vector.

Still another aspect of the present invention is directed to antibodies to B38, B55 and/or B60 or B38, B55 and/or B60 (herein referred to as “the immunogen”) including catalytic antibodies. Such antibodies may be monoclonal or polyclonal and may be selected from naturally occurring antibodies or may be specifically raised. In the case of the latter, the immunogen may first need to be associated with a carrier molecule. The antibodies of the present invention are particularly useful as therapeutic or diagnostic agents. Alternatively, fragments of antibodies may be used such as Fab fragments. Furthermore, the present invention extends to recombinant and synthetic antibodies and to antibody hybrids. A “synthetic antibody” is considered herein to include fragments and hybrids of antibodies. The antibodies of this aspect of the present invention are particularly useful for immunotherapy and may also be used as a diagnostic tool, for example, for monitoring the program of a therapeutic regime.

For example, specific antibodies can be used to screen for the immunogen. The latter would be important, for example, as a means for screening for levels of the immunogen in a cell extract or other biological fluid or purifying sphingosine kinase made by recombinant means from culture supernatant fluid. Techniques for the assays contemplated herein are known in the art and include, for example, sandwich assays, ELISA and flow cytometry.

It is within the scope of this invention to include any second antibodies (monoclonal, polyclonal or fragments of antibodies) directed to the first mentioned antibodies discussed above. Both the first and second antibodies may be used in detection assays or a first antibody may be used with a commercially available anti-immunoglobulin antibody. An antibody as contemplated herein includes any antibody specific to any region of the immunogen.

Both polyclonal and monoclonal antibodies are obtainable by immunization with the immunogen or derivatives and either type is utilizable for immunoassays. The methods of obtaining both types of sera are well known in the art. Polyclonal sera are less preferred but are relatively easily prepared by injection of a suitable laboratory animal with an effective amount of the immunogen or antigenic parts thereof, collecting serum from the animal, and isolating specific sera by any of the known immunoadsorbent techniques. Although antibodies produced by this method are utilizable in virtually any type of immunoassay, they are generally less favoured because of the potential heterogeneity of the product.

The use of monoclonal antibodies in an immunoassay is particularly preferred because of the ability to produce them. in large quantities and the homogeneity of the product. The preparation of hybridoma cell lines for monoclonal antibody production derived by fusing an immortal cell line and lymphocytes sensitized against the immunogenic preparation can be done by techniques which are well known to those who are skilled in the art. (See, for example Douillard and Hoffman, Basic Facts about Hybridomas, in Compendium of Immunology Vol II, ed. by Schwartz, 1981; Kohler and Milstein, Nature 256: 495-499, 1975; European Journal of Immunology 6: 511-519, 1976).

In another aspect of the present invention, the molecules of the present invention are also useful as screening targets for use in applications such as the diagnosis of disorders which are regulated by B38, B55 and/or B60 or B38, B55 and/or B60.

Yet another aspect of the present invention contemplates a method for detecting B38, B55 and/or B60 or B38, B55 and/or B60 mRNA in a biological sample from a subject said method comprising contacting said biological sample with an antibody specific for B38, B55 and/or B60 or B38, B55 and/or B60 mRNA or its derivatives or homologs for a time and under conditions sufficient for a complex to form, and then detecting said complex. Such methods may be particularly useful for the diagnosis of the development of or predisposition to obesity, anorexia, diabetes or energy imbalance.

The presence of B38, B55 and/or B60 may be determined in a number of ways such as by Western blotting, ELISA or flow cytometry procedures. B38, B55 and/or B60 mRNA may be detected, for example, by in situ hybridization or Northern blotting. These, of course, include both single-site and two-site or “sandwich” assays of the non-competitive types, as well as in the traditional competitive binding assays. These assays also include direct binding of a labelled antibody to a target.

Sandwich assays are among the most useful and commonly used assays and are favoured for use in the present invention. A number of variations of the sandwich assay technique exist, and all are intended to be encompassed by the present invention. Briefly, in a typical forward assay, an unlabelled antibody is immobilized on a solid substrate and the sample to be tested brought into contact with the bound molecule. After a suitable period of incubation, for a period of time sufficient to allow formation of an antibody-antigen complex, a second antibody specific to the antigen, labelled with a reporter molecule capable of producing a detectable signal is then added and incubated, allowing time sufficient for the formation of another complex of antibody-antigen-labelled antibody. Any unreacted material is washed away, and the presence of the antigen is determined by observation of a signal produced by the reporter molecule. The results may either be qualitative, by simple observation of the visible signal, or may be quantitated by comparing with a control sample containing known amounts of hapten. Variations on the forward assay include a simultaneous assay, in which both sample and labelled antibody are added simultaneously to the bound antibody. These techniques are well known to those skilled in the art, including any minor variations as will be readily apparent. In accordance with the present invention the sample is one which might contain B38, B55 and/or B60 or B38, B55 and/or B60 including cell extract, tissue biopsy or possibly serum, saliva, mucosal secretions, lymph, tissue fluid and respiratory fluid. The sample is, therefore, generally a biological sample comprising biological fluid but also extends to fermentation fluid and supernatant fluid such as from a cell culture.

In the typical forward sandwich assay, a first antibody having specificity for the B38, B55 and/or B60 or B38, B55 and/or B60 or antigenic parts thereof, is either covalently or passively bound to a solid surface. The solid surface is typically glass or a polymer, the most commonly used polymers being cellulose, polyacrylamide, nylon, polystyrene, polyvinyl chloride or polypropylene. The solid supports may be in the form of tubes, beads, discs of microplates, or any other surface suitable for conducting an immunoassay. The binding processes are well-known in the art and generally consist of cross-linking covalently binding or physically adsorbing, the polymer-antibody complex is washed in preparation for the test sample. An aliquot of the sample to be tested is then added to the solid phase complex and incubated for a period of time sufficient (e.g. 2-40 minutes) and under suitable conditions (e.g. 25° C.) to allow binding of any subunit present in the antibody. Following the incubation period, the antibody subunit solid phase is washed and dried and incubated with a second antibody specific for a portion of the hapten. The second antibody is linked to a reporter molecule which is used to indicate the binding of the second antibody to the hapten.

An alternative method involves immobilizing the target molecules in the biological sample and then exposing the immobilized target to specific antibody which may or may not be labelled with a reporter molecule. Depending on the amount of target and the strength of the reporter molecule signal, a bound target may be detectable by direct labelling with the antibody. Alternatively, a second labelled antibody, specific to the first antibody is exposed to the target-first antibody complex to form a target-first antibody-second antibody tertiary complex. The complex is detected by the signal emitted by the reporter molecule.

By “reporter molecule” as used in the present specification, is meant a molecule which, by its chemical nature, provides an analytically identifiable signal which allows the detection of antigen-bound antibody. Detection may be either qualitative or quantitative. The most commonly used reporter molecules in this type of assay are either enzymes, fluorophores or radionuclide containing molecules (i.e. radioisotopes) and chemiluminescent molecules.

In the case of an enzyme immunoassay, an enzyme is conjugated to the second antibody, generally by means of glutaraldehyde or periodate. As will be readily recognized, however, a wide variety of different conjugation techniques exist, which are readily available to the skilled artisan. Commonly used enzymes include horseradish peroxidase, glucose oxidase, beta-galactosidase and alkaline phosphatase, amongst others. The substrates to be used with the specific enzymes are generally chosen for the production, upon hydrolysis by the corresponding enzyme, of a detectable color change. Examples of suitable enzymes include alkaline phosphatase and peroxidase. It is also possible to employ fluorogenic substrates, which yield a fluorescent product rather than the chromogenic substrates noted above. In all cases, the enzyme-labelled antibody is added to the first antibody hapten complex, allowed to bind, and then the excess reagent is washed away. A solution containing the appropriate substrate is then added to the complex of antibody-antigen-antibody. The substrate will react with the enzyme linked to the second antibody, giving a qualitative visual signal, which may be further quantitated, usually spectrophotometrically, to give an indication of the amount of hapten which was present in the sample. “Reporter molecule” also extends to use of cell agglutination or inhibition of agglutination such as red blood cells on latex beads, and the like.

Alternately, fluorescent compounds, such as fluorecein and rhodamine, may be chemically coupled to antibodies without altering their binding capacity. When activated by illumination with light of a particular wavelength, the fluorochrome-labelled antibody adsorbs the light energy, inducing a state to excitability in the molecule, followed by emission of the light at a characteristic color visually detectable with a light microscope. As in the EIA, the fluorescent labelled antibody is allowed to bind to the first antibody-hapten complex. After washing off the unbound reagent, the remaining tertiary complex is then exposed to the light of the appropriate wavelength and the fluorescence observed indicates the presence of the hapten of interest. Immunofluorescene and EIA techniques are both very well established in the art and are particularly preferred for the present method. However, other reporter molecules, such as radioisotope, chemiluminescent or bioluminescent molecules, may also be employed.

The present invention also contemplates genetic assays such as involving PCR analysis to detect B38, B55 and/or B60 or its derivatives. Genetic assays directed to detecting B38, B55 and/or B60 have a wide variety of applications including, but not limited to, diagnosing disorders involving aberrant expression of one or more of these molecules or expression of specific polymorphic varients or isoforms of these molecules. Such assays may also be utilised to genetically screen individuals for the purpose of assessing the existence of a predisposition to the development of such disorders. For example, to detect the expression of given genetic polymorphic forms of any one of B38, B55 and/or B60, or the existence of specific haplotypes of these genes. In this regard, by determining gene expression patterns a mechanism is provided for designing treatment strategies appropriate for the subject individual.

The present invention should also be understood to extend to methods of diagnosing or monitoring a disease condition in a mammal, which disease condition is characterised by aberrant B38, B55 and/or B60 expression or functional activity, said method comprising screening for B38, B55 and/or B60 or B38, B55 and/or B60 in a biological sample from said mammal.

Further features of the present invention are more fully described in the following non-limiting Examples.

### Summary Of Sequence Id Nos

A summary of sequence identity numbers used throughout the subject specification are provided in Table 2.

TABLE 2

| SEQ ID NO: | DESCRIPTION |
|---|---|
| <400>1 | cDNA Nucleotide sequence of murine B38 |
| <400>2 | Amino acid sequence of murine B38 |
| <400>3 | cDNA Nucleotide sequence of murine B55 |
| <400>4 | Amino acid sequence of murine B55 |
| <400>5 | cDNA sequence of human B55 |
| <400>6 | Amino acid sequence of human B55 |
| <400>7 | cDNA Nucleotide sequence of murine B60 |
| <400>8 | Amino acid sequence of murine B60 |
| <400>9 | Genomic sequence of human B55 |
| <400>10 | Primer sequence |
| <400>11 | Primer sequence |
| <400>12 | Primer sequence |
| <400>13 | Primer sequence |
| <400>14 | Primer sequence |
| <400>15 | Primer sequence |
| <400>16 | Primer sequence |
| <400>17 | Primer sequence |
| <400>18 | Fluorogenic probe sequence |
| <400>19 | Fluorogenic probe sequence |
| <400>20 | Fluorogenic probe sequence |
| <400>21 | Fluorogenic probe sequence |
| <400>22 | Expressed sequence tag |

### Amino Acid Abbreviations

A summary of the single and three letter abbreviations for amino acid residues used in the present specification is provided in Table 3.

TABLE 3
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | The | T |
| Tryptophan | Trp | W |
| Tyrosine | Tyr | Y |
| Valine | Val | V |
| Any residue | Xaa | X |

### EXAMPLE 1

### Animals

A Psammomys obesus colony is maintained at Deakin University, with the breeding pairs fed ad libitum a diet of lucerne and chow. Experimental animals were weaned at four weeks of age and given a diet of standard laboratory chow from which 12% of energy was derived from fat, 63% from carbohydrate and 25% from protein (Barastoc, Pakenham, Australia). Animals were housed individually in a temperature controlled room (22±1° C.) with a 12-12-hour light-dark cycle. At 18 weeks of age, animals were sacrificed and the tissues immediately removed, frozen in liquid N₂ and then stored at −80° C.

For experimental purposes, Psammomys obesus can be classified into three groups according to their blood glucose and plasma insulin concentration, taken in the fed state at 16 weeks of age. Group A animals are normoglycemic (blood glucose <8.0 mmol/L) and normoinsulinemic (plasma insulin <150 mU/L), Group B animals are normoglycemic but hyperinsulinemic (plasma insulin >150 mU/I), and Group C animals are hyperglycemic (blood glucose >150 mU/I) and hyperinsulinemic. The criteria for the classification of animals into groups were based on those of Kalderon et al. 1986, who first characterized the stages of development of the obesity/diabetes syndrome in this species.

### EXAMPLE 2

### Sequencing and Cloning of B38, B55 and B60

B38, B55 and B60 were all identified by differential display PCR using the RNAimage mRNA differential display system (GenHunter Corporation). Liver mRNA from fed and fasted, lean and obese Psammomys obesus was compared. The PCR products were separated on a 6% polyacrylamide gel, and differentially expressed PCR fragments were visualized by exposing the dried gel to x-ray film. Candidate bands were exised from the gel and reamplified by PCR using the appropriate primer combination. Sequencing reactions were carried out using ABI PRISM Big-Dye terminator cycle sequencing ready reaction kits and analysed on an ABI 373A DNA sequencer. Gene database searches were performed at the National Centre for Biotechnology Information using the BLAST network service. In order to obtain the full mRNA sequence, 5′ and 3′ RACE (Rapid Amplification of cDNA Ends) was performed using the Marathon cDNA amplification kit (Clontech). The RACE PCR product was cloned into the pCR-TRAP cloning system (GenHunter Corporation). Finally, the genes were sequenced in the forward direction to confirm the sequence. Cloning of the RACE product was unsuccessful for B60, and so for this gene probing a cDNA library is necessary.

### EXAMPLE 3

### Expression Analysis

Liver and muscle RNA was extracted using RNAzol B (Tel-Test) and adipose tissue RNA using the Rneasy RNA extraction kit (Qiagen). The RNA was reverse transcribed with AMV (Promega) to form cDNA. The level of gene expression in each cDNA sample was quantitated using Taqman PCR technology on an ABI Prism 7700 sequence detector. β-actin was used as an endogenous control to standardise the amount of cDNA added to a reaction. Primer sequences were as follows:

B38 forward, 5′-GGGAGAGCTGTGGAGTCAACA-3′ [<400>10];
B38 reverse, 5′-CGTGGCGACTTAGTGTAGCATT-3′ [<400>11];
B55 forward, 5′-GATGCGTTCAATGATGTCTTCCT-3′ [<400>12];
B55 reverse, 5′-AGAAGCAAACCCCATCAACTGT-3′ [<400>13];
B60 forward, 5′-TGGAGGTTCTTCGATGCTCAT-3′ [<400>14];
B60 reverse, 5′-CAGTGAAACACGTCTGCTTCTG-3′ [<400>15];
β-actin forward, 5′-GCAAAGACCTGTATGCCAACAC-3′ [<400>16];
β-actin reverse, 5′-GCCAGAGCAGTGATCTCTTTCTG-3′ [<400>17];
Fluorogenic probe sequences were 5′-ACCGTGCTGCCCAGGTGTCCA-3′ [<400>18]
for B38;
5′-TGAGCCCACCAGTGAGGATTACTGATGTG-3′ [<400>19] for B55;
5′-ATCTTCTTTGAAGTGGAGTGGAGACGCTG-3′ [<400>20] for B60 and
5′-TCCGGTCCACAATGCCTGGGTACAT-3′ [<400>21] for β-actin.

The probes had the reporter dye FAM attached to the 5′ end and both probes had the quencher dye TAMRA attached to the 3′ end. PCR conditions were 50° C. for 2 min. 95° C. for 10 min followed by 40 cycles of 95° C. for 15 sec and 60° C. for 1 min.

### EXAMPLE 4

### B38

Sequence and Structure

The full sequence of the B38 transcript is 1669 nucleotides in length and encodes a protein of 354 amino acids. The protein sequence has regions of high homology to complement factor precursors C5 and C3. An 18 amino acid hydrophobic signal peptide is found at the amino terminal, which indicates that the protein is either secreted or has a transmembrane segment. However B38 is thought to be secreted since the signal sequence is very similar to that of C5 which is also secreted. One region of high homology is shared with C3a and C5a, which have an anaphylatoxin-like domain, and both of these factors have been shown to increase hepatic glucose output. Acylation stimulating protein (ASP) is a derivative of C3a and stimulates triglyceride synthesis and glucose transport in adipocytes. C3a and C5a are cleaved from the very large proteins C3 and C5, respectively, while B38 is a much smaller transcript.

Gene Expression

In the liver of Psammomys obesus, B38 mRNA levels positively correlate with body weight (p<0.01 with all animals together, and p<0.001, group B animals). There is also a positive correlation with triglycerides (p<0.05). No difference in the level of expression was seen in the liver between fed and fasted animals.

A positive correlation with triglycerides was also seen in the adipose tissue (p<0.02). Again, there was no significant different between fed and fasted groups. A positive correlation between B38 gene expression in the muscle and blood glucose levels was found (p<0.01) in lean and healthy (group A) animals. This was not seen in group B or C animals.

### EXAMPLE 5

### B55

Sequence and Structure

The B55 mRNA is 1155 nucleotides in length and does not match any known genes in the public database, but has homology with expressed sequence tags (ESTs) from a variety of tissues. The predicted open reading frame results in a protein of 189 amino acids in Psammomys obesus. Mouse, rat and human sequences were deduced from ESTs (3 rat, 5 mouse and 8 human sequences were used). The mouse and rat protein were found to be 188 amino acids long and were 91% and 93% homologous to the Psammomys obesus sequence, respectively. The human protein was found to be 187 amino acids long and was 82% homologous to the Psammomys obesus sequence. There were no nucleotide or amino acid differences found between lean, obese or diabetic Psammomys obesus. B55 is located on chromosome 15 in humans from 15q26.1 to 15qter and on chromosome 7 in mice.

B55 is predicted to have one transmembrane region at residues 37 to 53 with a C-terminal cytoplasmic tail. The tail contains a coiled coil region from amino acids 79 to 117. Coiled coil regions are found predominantly in some structural proteins and in a class of DNA-binding proteins in which the coiled coil region is called a leucine zipper domain. The coiled coil in B55 is only about 40 residues long, much shorter than the very long coils found in many fibrous proteins such as mysosin and keratin. It also does not appear to be a leucine zipper which are characterized by a leucine every seventh residue. There are 5 leucines, all of which are at a or d sites but they do not line up down one side of the helix. Coiled coils are found within many other proteins, however, and mediate a wide variety of functions.

A dileucine motif was also found in the cytoplasmic tail. Dileucine motifs have been shown to be involved in trans Golgi sorting, lysosomal targeting and internalization of a number of proteins. The insulin receptor, β2-adrenergic receptor and the glucose transporter GLUT4 all have a dileucine motif which is involved in internalization. B55 has one potential PEST sequence (RPQEEDGPGPSTSSSVTR <400>22). Proteins with intracellular half-lives of less than two hours are found to contain regions rich in proline, glutamic acid, serine and threonine (P, E, S and T). These so called PEST regions are generally flanked by clusters of positively charged amino acids.

Gene Expression

B55 gene expression was found to be significantly upregulated in the liver of fasted compared to fed animals (p<0.0001). This was evident in groups A, B and C, and the difference appeared more pronounced in obese, diabetic animals. A similar trend was observed in the adipose tissue, with higher levels of expression after fasting (p<0.05). This was found in groups B and C only, with the greatest difference in C animals.

In the fed state, there was a significant correlation between liver gene expression and blood triglyceride levels (p<0.01).

Cell Culture Studies

Glucose and insulin effects—HepG2 cells (grown in high glucose media) were treated with different concentrations of insulin (5 nM, 50 nM and 500 nM) for 4 or 24 hours. 4 hours of insulin treatment in high glucose media caused a dose-dependent decrease in B55 expression. Treatment with 5 nM insulin caused a 25% reduction in B55 expression whilst 50 nM and 500 nM insulin caused a 42%-43% reduction in expression. The decrease in B55 expression with insulin treatment was statistically significant at 50 nM and 500 nM (ANOVA, p<0.05) when compared to the untreated controls. A similar result was observed after 24 hours treatment with insulin (5 nM, 50 nM, 500 nM) in high glucose media. 5 nM insulin for 24 hours caused a 23% reduction in B55 gene expression whilst 50 nM and 500 nM insulin produced a 62% -63% reduction in expression.

### EXAMPLE 6

### B60

Sequence and Structure

A portion of the B60 sequence has been obtained. Only the 5′ end remains to be elucidated. The mRNA transcript sequence so far is 279 nucleotides long with the most likely reading frame giving a 28 amino acid protein. This protein appears to have a transmembrane segment and is possibly located in the endoplasmic reticulum.

Gene Expression

In the liver, B60 was seen to positively correlate with body weight (p<0.01 with all animals, p<0.05 A animals, p<0.02 B animals). In the fasted state, B60 expression in the muscle significantly correlates with body weight (p<0.05) and with insulin (p<0.00 1).

### EXAMPLE 7

### Human B55

The human ESTs used to determine the B55 cDNA sequence were (GenBank accession numbers in bold):

1. AA305753, Homo sapiens cDNA, Jurkat Tcells VI, Est 176916, 5′ end

2. N42213, Homo sapiens cDNA clone 257698, yw7le06.rl, 5′ end 3. AA885020, am4lcO8.sl Soares NFL T GBC SI Homo sapiens cDNA clone IMAGE:1471310, 3′ end.

4. AA629979, ae64fo5.sl Stratagene lung carcinoma 937218, Homo sapiens cDNA clone 951681, 3′ end.

5. AA330253, EST 33955, Embryo 12 wk II Homo sapiens cDNA, 5′ end.

6. AA364761, EST 75676, Pineal gland II, Homo sapiens cDNA, 5′ end.

7. N43740, YY 18603.R1 Soares melanocyte 2NbHM Homo Sapiens cDNA clone Image: 271565 5′.

8. H14102, ym62a01.r1 Soares adult brain N2b4HB55Y Homo sapiens cDNA clone IMAGE: 163464 5′.

The human genomic clone containing B55 was identified by a homology search of the B55 cDNA sequence against new additions to the NCBI GenBank database. The exon/intron structure was determined by first aligning the cDNA sequence to the genomic sequence and then applying the GT-AG rule to determine the exact boundaries. Introns almost invariantly begin with GT and end in AG.

The protein sequence was first deduced from the human cDNA sequence using the ExPASy Translate program, and then confirmed using this program with the genomic sequence once that became available.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Barnett M, Collier G R, Collier F M, Zimmet P, O'Dea K (1994a) A cross-sectional and short-term longitudinal characterisation of NIDDM in Psammomys obesus. Diabetologia 37: 671-676.

Barnett M, Collier GR, Zinunet P, O'Dea K (1994b) The effect of restricting energy intake on diabetes in Psammomys obesus. Int J Obesity 18: 789-794.

Bennett S A, Magnus P (1994) Trends in cardiovascular risk factors in Australia: Results from the National Heart Foundation's Risk Factor Prevalence Study, 1980-1989. Med J Aust 161: 519-527.

Bonner et al. (1973) J.Mol.Biol, 81:123.

Bouchard C. The genetics of obesity.Boca Raton: CRC Press, 1994.

Collier G R, de Silva A, Sanigorski A, Walder K, Yamamoto A, Zimmet P (1997a) Development of obesity and insulin resistance in the Israeli Sand Rat (Psammomys obesus): Does leptin play a role. Ann New York Acad Sci 827: 50-63.

Collier G R, Walder K, de Silva A, Morton G, Zimmet P (1997b) Diabetes, obesity and leptin in the Israeli Sand Rat (Psammomys obesus). Exp Clin Endocrinol Diabetes 105: 36-37.

DeFronzo R A (1988) The triumvirate B-cell, muscle and liver: A collusion responsible for NIDDM. Diabetes 37: 667-688.

Flegal, K. M., Carroll, M. D., Kuczmarski, R. J. and Johnson, C. L. (1998) Int J Obesity 22:39-47.

Harris, M. I. (1998) Diabetes Care 21 (Suppl. 3):C-11-C14.

Jonsson, B. (1998) Diabetes Care 21 (Suppl. 3):C7-C10.

Kalderon B., Gutman, A., Levy, A., Shafrir, E. and Adler, J. H. (1986) Diabetes 6:717-724.

Martikainen, P. T. and Marmot, M. G. (1999) Am J Clin Nutr 69:719-726.

Shafrir E, Gutman A (1993) Psammomys obesus of the Jerusalem colony: A model for nutritionally induced, non-insulin-dependent diabetes. J Basic Clin Physiol Pharm 4: 83-99.

Story, M., Evans, M., Fabsitz, R. R., Clay, T. E., Holy Rock, B. and Broussard, B. (1999) Am J Clin Nutr 69:747S-754S.

Walder K, Dascaliuc C R, Lewandowski P A, Sanigorski A J, Zimmet P, Collier G R (1997a) The effect of dietary energy restriction on the development of obesity and non-insulin-dependent diabetes mellitus (NIDDM) in Psammomys obesus. Obesity Res 5: 193-200.

Zhang Y, Proenca R, Maffei M, Barone M, Leopold L, Friedman J M (1994) Positional cloning of the mouse obese gene and its human homologue. Nature 372: 425-432.

1. An isolated nucleic acid molecule or derivative, homologue or analogue thereof comprising a nucleotide sequence encoding or complementary to a sequence encoding a protein or derivative, homologue or mimetic of said protein wherein said nucleic acid molecule is differentially expressed in liver tissue of obese animals compared to lean animals.

2. The isolated nucleic acid molecule according to claim 1 wherein said protein comprises the amino acid sequence substantially as set forth in SEQ ID NO:2 or a derivative, homologue or mimetic thereof or having at least about 45% similarity to at least 10 contiguous amino acids in SEQ ID NO:2.

3. The isolated nucleic acid molecule according to claim 2 comprising a nucleotide sequence substantially as set forth in SEQ ID NO:1 or a derivative or homologue thereof or capable of hybridising to SEQ ID NO:1 under low stringency conditions.

4. The isolated nucleic acid molecule according to claim 3 which further encodes an amino acid sequence corresponding to an amino acid sequence set forth in SEQ ID NO:2 or a sequence having at least about 45% similarity to at least 10 contiguous amino acids in SEQ ID NO:2.

5. The isolated nucleic acid molecule according to claim 2 substantially as set forth in SEQ ID NO:1.

6. The isolated nucleic acid molecule according to claim 1 wherein said protein comprises the amino acid sequence substantially as set forth in SEQ ID NO:4 or a derivative, homologue or mimetic thereof or having at least about 45% similarity to at least 10 contiguous amino acids in SEQ ID NO:4.

7. The isolated nucleic acid molecule according to claim 6 comprising a nucleotide sequence substantially as set forth in SEQ ID NO:3 or a derivative or homologue thereof or capable of hybridising to SEQ ID NO:3 under low stringency conditions.

8. The isolated nucleic acid molecule according to claim 7 which further encodes an amino acid sequence corresponding to an amino acid sequence set forth in SEQ ID NO:4 or a sequence having at least about 45% similarity to at least 10 contiguous amino acids in SEQ ID NO:4.

9. The isolated nucleic acid molecule according to claim 6 substantially as set forth in SEQ ID NO:3.

10. The isolated nucleic acid molecule according to claim 1 wherein said protein comprises the amino acid sequence substantially as set forth in SEQ ID NO:6 or a derivative, homologue or mimetic thereof or having at least about 45% similarity to at least 10 contiguous amino acids in SEQ ID NO:6.

11. The isolated nucleic acid molecule according to claim 10 comprising a nucleotide sequence substantially as set forth in SEQ ID NO:5 or a derivative or homologue thereof or capable of hybridising to SEQ ID NO:5 under low stringency conditions.

12. The isolated nucleic acid molecule according to claim 11 which further encodes an amino acid sequence corresponding to an amino acid sequence set forth in SEQ ID NO:6 or a sequence having at least about 45% similarity to at least 10 contiguous amino acids in SEQ ID NO:6.

13. The isolated nucleic acid molecule according to claim 10 substantially as set forth in SEQ ID NO:5.

14. The isolated nucleic acid molecule according to claim 1 wherein said protein comprises the amino acid sequence substantially as set forth in SEQ ID NO:8 or a derivative, homologue or mimetic thereof or having at least about 45% similarity to at least 10 contiguous amino acids in SEQ ID NO:8.

15. The isolated nucleic acid molecule according to claim 14 comprising a nucleotide sequence substantially as set forth in SEQ ID NO:7 or a derivative or homologue thereof or capable of hybridising to SEQ ID NO:7 under low stringency conditions.

16. The isolated nucleic acid molecule according to claim 15 which further encodes an amino acid sequence corresponding to an amino acid sequence set forth in SEQ ID NO:8 or a sequence having at least about 45% similarity to at least 10 contiguous amino acids in SEQ ID NO:8.

17. The isolated nucleic acid molecule according to claim 14 substantially as set forth in SEQ ID NO:7.

18. The isolated nucleic acid molecule according to claim 1 comprising a nucleotide sequence substantially as set forth in SEQ ID NO:9 or a derivative or homologue thereof or capable of hybridising to SEQ ID NO:9 under low stringency conditions.

19. The isolated nucleic acid molecule according to claim 18 which further encodes an amino acid sequence corresponding to an amino acid sequence set forth in SEQ ID NO:6 or a sequence having at least about 45% similarity to at least 10 contiguous amino acids in SEQ ID NO:6.

20. The isolated nucleic acid molecule according to claim 18 substantially as set forth in SEQ ID NO:9.

21. An isolated protein or a derivative, homologue, analogue, chemical equivalent or mimetic thereof wherein said protein is differentially expressed in liver tissue of obese animals compared to lean animals.

22. The isolated protein according to claim 21 comprising an amino acid sequence substantially as set forth in SEQ ID NO:2 or a derivative, homologue or mimetic thereof or a sequence having at least about 45% similarity to at least 10 contiguous amino acids in SEQ ID NO:2 or a derivative, homologue, analogue, chemical equivalent or mimetic of said protein.

23. The isolated protein according to claim 22 encoded by a nucleotide sequence substantially as set forth in SEQ ID NO:1 or a derivative, homologue or analogue thereof or capable of hybridising to SEQ ID NO:1 under low stringency conditions or a derivative, homologue, analogue, chemical equivalent or mimetic of said protein.

24. The isolated protein according to claim 23 substantially as set forth in SEQ ID NO:2.

25. The isolated protein according to claim 21 comprising an amino acid sequence substantially as set forth in SEQ ID NO:4 or a derivative, homologue or mimetic thereof or a sequence having at least about 45% similarity to at least 10 contiguous amino acids in SEQ ID NO:4 or a derivative, homologue, analogue, chemical equivalent or mimetic of said protein.

26. The isolated protein according to claim 25 encoded by a nucleotide sequence substantially as set forth in SEQ ID NO:3 or a derivative, homologue or analogue thereof or capable of hybridising to SEQ ID NO:3 under low stringency conditions or a derivative, homologue, analogue, chemical equivalent or mimetic of said protein.

27. The isolated protein according to claim 25 substantially as set forth in SEQ ID NO:4.

28. The isolated protein according to claim 21 comprising an amino acid sequence substantially as set forth in SEQ ID NO:6 or a derivative, homologue or mimetic thereof or a sequence having at least about 45% similarity to at least 10 contiguous amino acids in SEQ ID NO:6 or a derivative, homologue, analogue, chemical equivalent or mimetic of said protein.

29. The isolated protein according to claim 28 encoded by a nucleotide sequence substantially as set forth in SEQ ID NO:5 or a derivative, homologue or analogue thereof or capable of hybridising to SEQ ID NO:5 under low stringency conditions or a derivative, homologue, analogue, chemical equivalent or mimetic of said protein.

30. The isolated protein according to claim 28 substantially as set forth in SEQ ID NO:6.

31. The isolated protein according to claim 21 comprising an amino acid sequence substantially as set forth in SEQ ID NO:8 or a derivative, homologue or mimetic thereof or a sequence having at least about 45% similarity to at least 10 contiguous amino acids in SEQ ID NO:8 or a derivative, homologue, analogue, chemical equivalent or mimetic of said protein.

32. The isolated protein according to claim 31 encoded by a nucleotide sequence substantially as set forth in SEQ ID NO:7 or a derivative, homologue or analogue thereof or capable of hybridising to SEQ ID NO:7 under low stringency conditions or a derivative, homologue, analogue, chemical equivalent or mimetic of said protein.

33. The isolated protein according to claim 31 substantially as set forth in SEQ ID NO:8.

34. The isolated protein according to claim 21 encoded by a nucleotide sequence substantially as set forth in SEQ ID NO:9 or a derivative, homologue or analogue thereof or capable of hybridising to SEQ ID NO:9 under low stringency conditions or a derivative, homologue, analogue, chemical equivalent or mimetic of said protein.

35. The isolated protein according to claim 21 wherein said protein is a homodimer.

36. The isolated protein according to claim 21 wherein said protein is a heterodimer.

37. A method of modulating expression of B38, B55 and/or B60 in a mammal, said method comprising contacting the B38, B55 and/or B60 gene with an effective amount of an agent for a time and under conditions sufficient to up-regulate, down-regulate or otherwise modulate expression of B38, B55 and/or B60.

38. A method of modulating activity of B38, B55 and/or B60 in a subject, said method comprising administering to said subject a modulating effective amount of an agent for a time and under conditions sufficient to increase or decrease B38, B55 and/or B60 activity.

39. A method of treating a mammal suffering from a condition characterised by one or more symptoms of obesity, anorexia, diabetes and/or energy imbalance said method comprising administering to said mammal an effective amount of an agent for a time and under conditions sufficient to modulate the expression of B38, B55 and/or B60 or sufficient to modulate the activity of B38, B55 and/or B60.

40. A method of treating a mammal suffering from a disease condition characterised by one or more symptoms of obesity, anorexia, diabetes or energy imbalance said method comprising administering to said mammal an effective amount of a protein according to claim 21.

41. A pharmaceutical composition comprising B38, B55 and/or B60, B38, B55 and/or B60 or an agent capable of modulating B38, B55 and/or B60 expression or B38, B55 and/or B60 activity together with one or more pharmaceutically acceptable carriers and/or diluents.

42. An isolated antibody directed to the protein according to claim 21.

43. An isolated antibody directed to the nucleic acid molecule according to claim 1.

44. The antibody according to claim 42 wherein said antibody is a monoclonal antibody.

45. The antibody according to claim 42 wherein said antibody is a polyclonal antibody.

46. A method for detecting B38, B55 and/or B60 in a biological sample from a subject said method comprising contacting said biological sample with an antibody specific for B38, B55 and/or B60 or its derivatives or homologues for a time and under conditions sufficient for a complex to form and then detecting said complex.

47. A method for detecting B38, BSS and/or B60 mRNA in a biological sample from a subject said method comprising contacting said biological sample with an antibody specific for B38, B55 and/or B60 mRNA or its derivatives or homologues for a time and under conditions sufficient for a complex to form and then detecting said complex.

48. A method of diagnosing and monitoring a mammalian disease condition, which disease condition is characterised by aberrant B38, B55 and/or B60 expression or functional activity, said method comprising screening for B38, B55 and/or B60 or B38, B55, and/or B60 in a biological sample form said mammal.

49. A method of diagnosing or monitoring a disease condition, which disease condition is characterised by one or more sympotoms of obesity, anorexia, diabetes and/or energy imbalance said method comprising screening for B38, B55 and/or B60 or B38, B55 and/or B60 would reduce all homologues thereof in the biological sample from said mammal.

50. An isolated nucleic acid molecule or derivative, homologue or analogue thereof comprising a nucleotide sequence encoding or complementary to a sequence encoding a protein or derivative, homologue or mimetic of said protein wherein said nucleic acid molecule is differentially expressed in liver tissue of fed animals compared to fasted animals.

51. The isolated nucleic acid molecule according to claim 50 wherein said protein comprises the amino acid sequence substantially as set forth in SEQ ID NO:2 or a derivative, homologue or mimetic thereof or having at least about 45% similarity to at least 10 contiguous amino acids in SEQ ID NO:2.

52. The isolated nucleic acid molecule according to claim 51 comprising a nucleotide sequence substantially as set forth in SEQ ID NO:1 or a derivative or homologue thereof or capable of hybridising to SEQ ID NO:1 under low stringency conditions.

53. The isolated nucleic acid molecule according to claim 52 which further encodes an amino acid sequence corresponding to an amino acid sequence set forth in SEQ ID NO:2 or a sequence having at least about 45% similarity to at least 10 contiguous amino acids in SEQ ID NO:2.

54. The isolated nucleic acid molecule according to claim 50 substantially as set forth in SEQ ID NO:1.

55. The isolated nucleic acid molecule according to claim 50 wherein said protein comprises the amino acid sequence substantially as set forth in SEQ ID NO:4 or a derivative, homologue or mimetic thereof or having at least about 45% similarity to at least 10 contiguous amino acids in SEQ ID NO:4.

56. The isolated nucleic acid molecule according to claim 55 comprising a nucleotide sequence substantially as set forth in SEQ ID NO:3 or a derivative or homologue thereof or capable of hybridising to SEQ ID NO:3 under low stringency conditions.

57. The isolated nucleic acid molecule according to claim 56 which further encodes an amino acid sequence corresponding to an amino acid sequence set forth in SEQ ID NO:4 or a sequence having at least about 45% similarity to at least 10 contiguous amino acids in SEQ ID NO:4.

58. The isolated nucleic acid molecule according to claim 55 substantially as set forth in SEQ ID NO:3.

59. The isolated nucleic acid molecule according to claim 50 wherein said protein comprises the amino acid sequence substantially as set forth in SEQ ID NO:6 or a derivative, homologue or mimetic thereof or having at least about 45% similarity to at least 10 contiguous amino acids in SEQ ID NO:6.

60. The isolated nucleic acid molecule according to claim 59 comprising a nucleotide sequence substantially as set forth in SEQ ID NO:5 or a derivative or homologue thereof or capable of hybridising to SEQ ID NO:5 under low stringency conditions.

61. The isolated nucleic acid molecule according to claim 60 which further encodes an amino acid sequence corresponding to an amino acid sequence set forth in SEQ ID NO:6 or a sequence having at least about 45% similarity to at least 10 contiguous amino acids in SEQ ID NO:6.

62. The isolated nucleic acid molecule according to claim 59 substantially as set forth in SEQ ID NO:5.

63. The isolated nucleic acid molecule according to claim 50 wherein said protein comprises the amino acid sequence substantially as set forth in SEQ ID NO:8 or a derivative, homologue or mimetic thereof or having at least about 45% similarity to at least 10 contiguous amino acids in SEQ ID NO:8.

64. The isolated nucleic acid molecule according to claim 63 comprising a nucleotide sequence substantially as set forth in SEQ ID NO:7 or a derivative or homologue thereof or capable of hybridising to SEQ ID NO:7 under low stringency conditions.

65. The isolated nucleic acid molecule according to claim 64 which further encodes an amino acid sequence corresponding to an amino acid sequence set forth in SEQ ID NO:8 or a sequence having at least about 45% similarity to at least 10 contiguous amino acids in SEQ ID NO:8.

66. The isolated nucleic acid molecule according to claim 63 substantially as set forth in SEQ ID NO:7.

67. The isolated nucleic acid molecule according to claim 50 comprising a nucleotide sequence substantially as set forth in SEQ ID NO:9 or a derivative or homologue thereof or capable of hybridising to SEQ ID NO:9 under low stringency conditions.

68. The isolated nucleic acid molecule according to claim 67 which further encodes an amino acid sequence corresponding to an amino acid sequence set forth in SEQ ID NO:6 or a sequence having at least about 45% similarity to at least 10 contiguous amino acids in SEQ ID NO:6.

69. The isolated nucleic acid molecule according to claim 67 substantially as set forth in SEQ ID NO:9.

70. An isolated protein or a derivative, homologue, analogue, chemical equivalent or mimetic thereof wherein said protein is differentially expressed in liver tissue of fed animals compared to fasted animals.

71. The isolated protein according to claim 70 comprising an amino acid sequence substantially as set forth in SEQ ID NO:2 or a derivative, homologue or mimetic thereof or a sequence having at least about 45% similarity to at least 10 contiguous amino acids in SEQ ID NO:2 or a derivative, homologue, analogue, chemical equivalent or mimetic of said protein.

72. The isolated protein according to claim 71 encoded by a nucleotide sequence substantially as set forth in SEQ ID NO:1 or a derivative, homologue or analogue thereof or capable of hybridising to SEQ ID NO:1 under low stringency conditions or a derivative, homologue, analogue, chemical equivalent or mimetic of said protein.

73. The isolated protein according to claim 72 substantially as set forth in SEQ ID NO:2.

74. The isolated protein according to claim 70 comprising an amino acid sequence substantially as set forth in SEQ ID NO:4 or a derivative, homologue or mimetic thereof or a sequence having at least about 45% similarity to at least 10 contiguous amino acids in SEQ ID NO:4 or a derivative, homologue, analogue, chemical equivalent or mimetic of said protein.

75. The isolated protein according to claim 74 encoded by a nucleotide sequence substantially as set forth in SEQ ID NO:3 or a derivative, homologue or analogue thereof or capable of hybridising to SEQ ID NO:3 under low stringency conditions or a derivative, homologue, analogue, chemical equivalent or mimetic of said protein.

76. The isolated protein according to claim 74 substantially as set forth in SEQ ID NO:4.

77. The isolated protein according to claim 70 comprising an amino acid sequence substantially as set forth in SEQ ID NO:6 or a derivative, homologue or mimetic thereof or a sequence having at least about 45% similarity to at least 10 contiguous amino acids in SEQ ID NO:6 or a derivative, homologue, analogue, chemical equivalent or mimetic of said protein.

78. The isolated protein according to claim 77 encoded by a nucleotide sequence substantially as set forth in SEQ ID NO:5 or a derivative, homologue or analogue thereof or capable of hybridising to SEQ ID NO:5 under low stringency conditions or a derivative, homologue, analogue, chemical equivalent or mimetic of said protein.

79. The isolated protein according to claim 77 substantially as set forth in SEQ ID NO:6.

80. The isolated protein according to claim 70 comprising an amino acid sequence substantially as set forth in SEQ ID NO:8 or a derivative, homologue or mimetic thereof or a sequence having at least about 45% similarity to at least 10 contiguous amino acids in SEQ ID NO:8 or a derivative, homologue, analogue, chemical equivalent or mimetic of said protein.

81. The isolated protein according to claim 80 encoded by a nucleotide sequence substantially as set forth in SEQ ID NO:7 or a derivative, homologue or analogue thereof or capable of hybridising to SEQ ID NO:7 under low stringency conditions or a derivative, homologue, analogue, chemical equivalent or mimetic of said protein.

82. The isolated protein according to claim 80 substantially as set forth in SEQ ID NO:8.

83. The isolated protein according to claim 70 encoded by a nucleotide sequence substantially as set forth in SEQ ID NO:9 or a derivative, homologue or analogue thereof or capable of hybridising to SEQ ID NO:9 under low stringency conditions or a derivative, homologue, analogue, chemical equivalent or mimetic of said protein.

84. The isolated protein according to claim 70 wherein said protein is a homodimer.

85. The isolated protein according to claim 70 wherein said protein is a heterodimer.

86. A method of treating a mammal suffering from a disease condition characterised by one or more symptoms of obesity, anorexia, diabetes or energy imbalance said method comprising administering to said mammal an effective amount of a nucleotide sequence according to claim 1.

87. An isolated antibody directed to the protein according to claim 70.

88. The antibody according to claim 87 wherein said antibody is a monoclonal antibody.

89. The antibody according to claim 87 wherein said antibody is a polyclonal antibody.
